(19) United States
(12) Patent Application Publication
(10) Pub. No.: US 2010/0019663 A1
(43) Pub. Date: Jan. 28, 2010

(54) FUSED AROMATIC COMPOUND AND ORGANIC LIGHT EMITTING DIODE COMPRISING ORGANIC LAYER COMPRISING THE SAME

(76) Inventors: Dong-woo SHIN, Seoul (KR); Byoung-ki CHOI, Hwaseong-si (KR); Myeong-suk KIM, Hwaseong-si (KR); Tae-yong NOH, Seoul (KR); O-hyun KWON, Seoul (KR); Tae-woo LEE, eoul (KR); Yu-jin KIM, Suwon-si (KR)

Correspondence Address: F. CHAU & ASSOCIATES, LLC, 130 WOODBURY ROAD, WOODBURY, NY, 11797, US

(21) Appl. No.: 12/480,206

(22) Filed: Jun. 8, 2009

(30) Foreign Application Priority Data
Jul. 24, 2008 (KR) 10-2008-0072436

(51) Int. Cl. H01J 1/63; C07D 209/80

(52) U.S. Cl. 313/504; 548/420

(57) ABSTRACT

Provided is a fused aromatic compound suitable for an organic layer of an organic light emitting emitting diode (OLED).

### CROSS-REFERENCE TO RELATED PATENT APPLICATION

This application claims the benefit of Korean Patent Application No. 10-2008-0072436, filed on Jul. 24, 2008, the disclosure of which is hereby incorporated by reference herein in its entirety.

### BACKGROUND OF THE INVENTION

1. Technical Field

The present disclosure relates to an organic layer of an organic light emitting diode, and more particularly, to an fused aromatic compound suitable for an emission layer and an organic light emitting diode including an organic layer including the fused aromatic compound.

2. Description of the Related Art

Organic light emitting diodes (OLEDs) have beneficial brightness, driving voltage, and quick response characteristics, and can realize multi color displays. Due to the above-mentioned benefits, a large amount of research into OLEDs has been carried out.

Typically, an OLED has an anode/organic emission layer/cathode structure. OLEDs can also have various other structures, such as an anode/hole injection layer (HIL)/hole transport layer (HTL)/emission layer (EML)/electron transport layer (ETL)/electron injection layer (EIL)/cathode structure or an anode/HIL/HTL/EML/hole blocking layer (HBL)/ETL/EIL/cathode structure.

A vacuum system may be necessary for manufacturing an OLED by vacuum deposition, and a shadow mask may be necessary to manufacture pixels for natural color displays. Meanwhile, when a solution coating method such as inkjet printing, screen printing, or spin coating is used, an organic layer can be readily and inexpensively manufactured and can have beneficial resolution properties.

Thus, there is a need in the art for a compound having beneficial properties and which is suitable for an organic layer interposed between a pair of electrodes of an OLED regardless of the method of forming the organic layer described above.

### SUMMARY OF THE INVENTION

In accordance with an exemplary embodiment of the present invention, there a fused aromatic compound represented by Formula 1 below is provided:

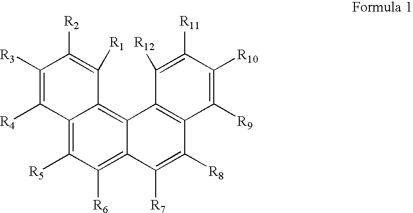

wherein R₁ to R₁₂ may be each independently a hydrogen atom, a halogen atom, a cyano group, a nitro group, a hydroxyl group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₅-C₆₀ cycloalkyl group, a substituted or unsubstituted C₅-C₆₀ cycloalkenyl group, a substituted or unsubstituted C₅-C₆₀ aryl group, a substituted or unsubstituted C₁-C₆₀ heteroaryl group, or a group represented by Formula 2 below, wherein at least one of R₁ to R₁₂ is the group represented by Formula 2:

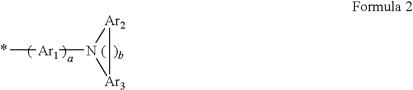

wherein Ar₁ may be a substituted or unsubstituted C₅-C₆₀ arylene group;

Ar₂ and Ar₃ may be each independently a substituted or unsubstituted C₅-C₆₀ aryl group;

a is an integer of 0 to 6; and

b is 0 or 1.

In accordance with an exemplary embodiment of the present invention, a fused aromatic compound represented by Formula 3 below is provided:

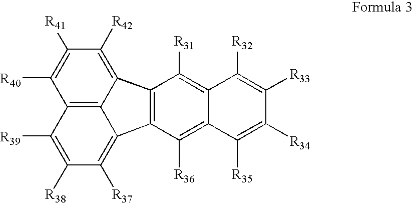

wherein R₃₁ to R₃₆ may be each independently a hydrogen atom, a halogen atom, a cyano group, a nitro group, a hydroxyl group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₅-C₆₀ cycloalkyl group, a substituted or unsubstituted C₅-C₆₀ cycloalkenyl group, a substituted or unsubstituted C₅-C₆₀ aryl group, a substituted or unsubstituted C₁-C₆₀ heteroaryl group, a group represented by Formula 4 below, or a group represented by Formula 5 below; and

R₃₇ to R₄₂ may be each independently a hydrogen atom or the group represented by Formula 5;

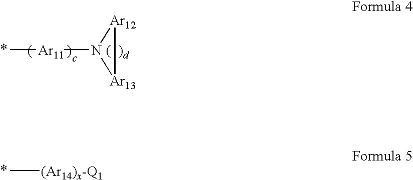

wherein Ar₁₁ and Ar₁₄ may be each independently a C₅-C₆₀ arylene group, or a C₅-C₆₀ arylene group substituted with at lest one selected from the group consisting of a halogen atom, a cyano group, a nitro group, a hydroxyl group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₅-C₆₀ cycloalkyl group, a substituted or unsubstituted C₅-C₆₀ cycloalkenyl group, a substituted or unsubstituted C₅-C₆₀ aryl group, a substituted or unsubstituted C₁-C₆₀ heteroaryl group, and —Si(R₄₄)(R₄₅)(R₄₆);

Ar₁₂ and Ar₁₃ may be each independently a C₅-C₁₄ aryl group, a C₅-C₁₄ aryl group substituted with at least one C₁-C₁₀ alkyl group, a C₅-C₁₄ aryl group substituted with at least one C₅-C₁₄ aryl group, or a C₅-C₁₄ aryl group substituted with at least one —Si(R₄₄)(R₄₅)(R₄₆); and

Q₁ is a hydrogen atom or —Si(R₄₇)(R₄₈)(R₄₉), wherein R₄₄ to R₄₉ may be each independently a hydrogen atom, a C₁-C₁₀ alkyl group, or a C₅-C₁₄ aryl group;

c is an integer of 1 to 6;

d is 0 or 1; and

x is an integer of 0 to 6,
1) except for when all of R₃₁ to R₄₂ are hydrogen atoms,
2) when all of R₃₇ to R₄₂ are hydrogen atoms, R₃₁ to R₃₆ are each independently a hydrogen atom, a group represented by Formula 4, or a group represented by Formula 5, provided that at least one of R₃₁ to R₃₆ is the group represented by Formula 4, or the group represented by Formula 5 wherein Q₁ is —Si(R₄₇)(R₄₈)(R₄₉), and

when all of R₃₁ to R₃₆ are hydrogen atoms, at least one of R₃₇ to R₄₂ is the group represented by Formula 5 wherein Q₁ is —Si(R₄₇)(R₄₈)(R₄₉).

In accordance with an exemplary embodiment of the present invention, a fused aromatic compound represented by Formula 6 below is provided:

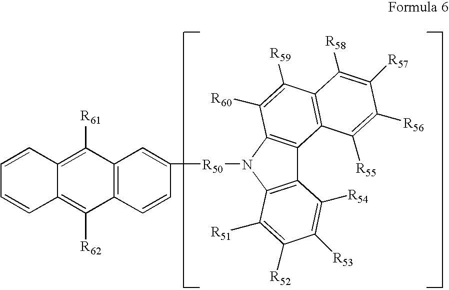

wherein one of R₅₀ to R₆₀ may be bonded to an anthracene ring of Formula 6;

R₅₀ to R₆₀ may be each independently a hydrogen atom, a halogen atom, a cyano group, a nitro group, a hydroxyl group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₅-C₆₀ cycloalkyl group, a substituted or unsubstituted C₅-C₆₀ cycloalkenyl group, a substituted or unsubstituted C₅-C₆₀ aryl group, a substituted or unsubstituted C₁-C₆₀ heteroaryl group, or —Si(R₆₅)(R₆₆)(R₆₇), wherein at least two adjacent groups among R₅₀ to R₆₀ are bonded to each other to form a ring;

R₆₁ and R₆₂ may be each independently a substituted or unsubstituted C₁-C₆₀ alkyl group, or a phenyl group; and

R₆₅ to R₆₇ may be each independently a hydrogen atom, a C₁-C₁₀ alkyl group, a C₂-C₁₀ alkenyl group, or a C₅-C₁₄ aryl group.

In accordance with an exemplary embodiment of the present invention, a fused aromatic compound represented by Formula 7 below is provided:

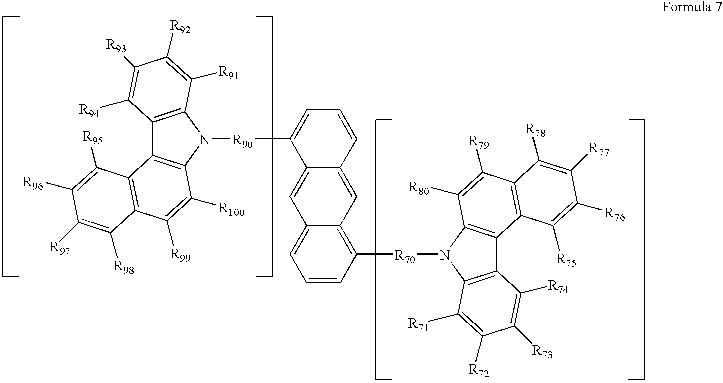

wherein one of R₇₀ to R₈₀ and one of R₉₀ to R₁₀₀ may be bonded to an anthracene ring of Formula 7;

R₇₀ to R₈₀ and R₉₀ to R₁₀₀ may be each independently a hydrogen atom, a halogen atom, a cyano group, a nitro group, a hydroxyl group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₅-C₆₀ cycloalkyl group, a substituted or unsubstituted C₅-C₆₀ cycloalkenyl group, a substituted or unsubstituted C₅-C₆₀ aryl group, a substituted or unsubstituted C₁-C₆₀ heteroaryl group, or —Si(R₁₀₁)(R₁₀₂)(R₁₀₃), wherein at least two adjacent groups among R₇₀ to R₈₀ and R₉₀ to R₁₀₀ are bonded to each other to form a ring; and

R₁₀₁ to R₁₀₃ may be each independently a hydrogen atom, a C₁-C₁₀ alkyl group, a C₂-C₁₀ alkenyl group, or a C₅-C₁₄ aryl group.

In accordance with another exemplary embodiment of the present invention, an organic light emitting diode (OLED) is provided. The OLED includes a substrate, a first electrode, a second electrode, and an organic layer interposed between the first electrode and the second electrode, wherein the organic layer may include a compound of any one of claims 1, 6, 13 and 17.

### BRIEF DESCRIPTION OF THE DRAWINGS

Exemplary embodiments of the present invention can be understood in more detail from the following description taken in conjunction with the attached drawings in which:

FIGS. 1A to 1C schematically illustrate organic light emitting diodes (OLEDs) according to an exemplary embodiment of the present invention;

FIG. 2 is a graph illustrating UV absorption spectra of solutions of Compounds 2 and 15 according to an exemplary embodiment of the present invention;

FIG. 3 is a graph illustrating photoluminescence (PL) spectra of Compounds 2 and 15 according to an exemplary embodiment of the present invention; and

FIG. 4 is a graph illustrating voltage-brightness characteristics of Sample 1 according to an exemplary embodiment of the present invention.

### DETAILED DESCRIPTION OF THE EXEMPLARY EMBODIMENTS OF THE INVENTION

Hereinafter, the present invention will be described more fully with reference to the accompanying drawings, in which exemplary embodiments of the invention are shown.

An fused aromatic compound according to an embodiment of the present invention may be represented by, for example, Formula 1 below.

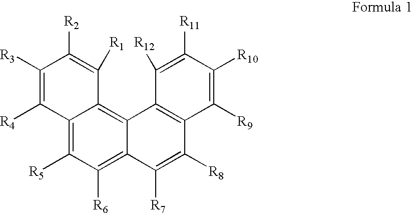

In Formula 1, R₁ to R₁₂ may, for example, be each independently a hydrogen atom, a halogen atom, a cyano group, a nitro group, a hydroxyl group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₅-C₆₀ cycloalkyl group, a substituted or unsubstituted C₅-C₆₀ cycloalkenyl group, a substituted or unsubstituted C₅-C₆₀ aryl group, a substituted or unsubstituted C₁-C₆₀ heteroaryl group, or a group represented by Formula 2 below.

For example,, R₁ to R₁₂ may be each independently a hydrogen atom, a halogen atom, a cyano group, a nitro group, a hydroxyl group, a substituted or unsubstituted C₁-C₁₀ alkyl group, a substituted or unsubstituted C₂-C₁₀ alkenyl group, a substituted or unsubstituted C₂-C₁₀ alkynyl group, a substituted or unsubstituted C₁-C₁₀ alkoxy group, a substituted or unsubstituted C₅-C₁₄ cycloalkyl group, a substituted or unsubstituted C₅-C₁₄ cycloalkenyl group, a substituted or unsubstituted C₅-C₁₄ aryl group, a substituted or unsubstituted C₁-C₁₄ heteroaryl group, or a group represented by Formula 2 below. More particularly, R₁ to R₁₂ may be each independently a hydrogen atom, a C₁-C₁₀ alkyl group, a C₂-C₁₀ alkenyl group, a C₅-C₁₄ aryl group, a C₅-C₁₄ aryl group substituted with at least one C₁-C₁₀ alkyl group, a C₅-C₁₄ aryl group substituted with at least one —Si(R₂₁)(R₂₂)(R₂₃), or the group represented by Formula 2, but are not limited thereto.

In Formula 1, at least one of R₁ to R₁₂ is the group represented by, for example, Formula 2 below.

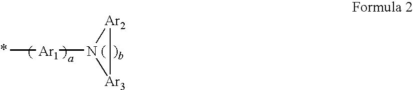

In Formula 2, Ar₁ may be, for example, a substituted or unsubstituted C₅-C₆₀ arylene group, Ar₂ to Ar₃ may be each independently a substituted or unsubstituted C₅-C₆₀ aryl group, a may be an integer of 0 to 6, and b may be 0 or 1. When a is 0, N of Formula 2 is directly bonded to a benzophenanthrene ring of Formula 1. Meanwhile, when b is 0, Ar₂ is not bonded to Ar₃ via a single bond, and when b is 1, Ar₂ is bonded to Ar₃ via a single bond.

Particularly, Ar₁ may be a substituted or unsubstituted C₅-C₁₄ arylene group, and more particularly a C₅-C₁₄ arylene group, a C₅-C₁₄ arylene group substituted with at least one C₁-C₁₀ alkyl group, a C₅-C₁₄ arylene group substituted with at least one C₅-C₁₄ aryl group, or a C₅-C₁₄ arylene group substituted with at least one —Si(R₂₁)(R₂₂)(R₂₃), but is not limited thereto.

In addition, Ar₂ and Ar₃ may be each independently a substituted or unsubstituted C₅-C₁₄ aryl group, and particularly each independently a C₅-C₁₄ aryl group, a C₅-C₁₄ aryl group substituted with at least one C₁-C₁₀ alkyl group, a C₅-C₁₄ aryl group substituted with at least one C₅-C₁₄ aryl group, or a C₅-C₁₄ aryl group substituted with at least one —Si(R₂₁)(R₂₂)(R₂₃), but are not limited thereto.

Particularly, R₂₁ to R₂₃ may be each independently a hydrogen atom, a C₁-C₁₀ alkyl group, or a substituted or unsubstituted C₅-C₁₄ aryl group, but are not limited thereto.

For example, the compound of Formula 1 may be represented by Formula 1a.

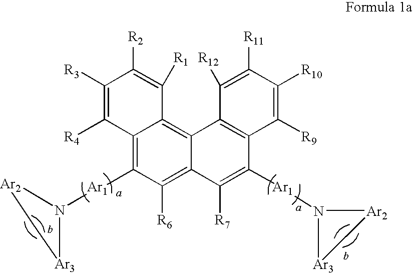

In Formula 1, R₅ and R₈ may be the group represented by Formula 2.

In Formula 2, the group represented by

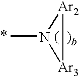

may be represented by one of Formulae 2a to 2k, but is not limited thereto.

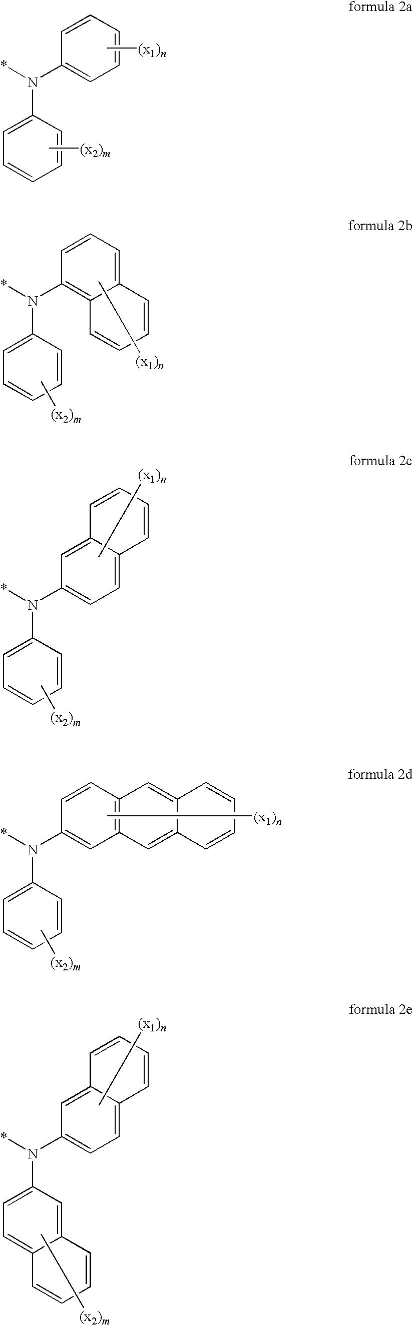

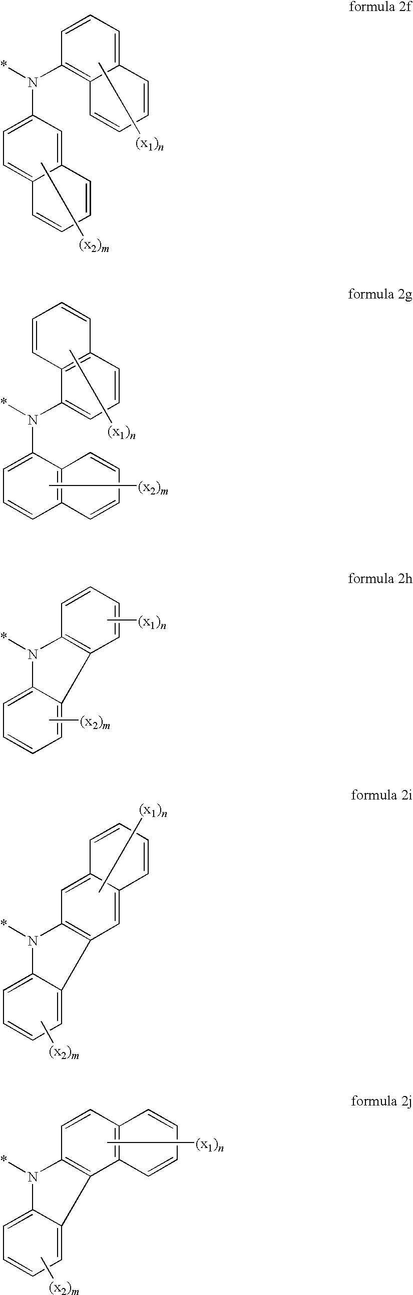

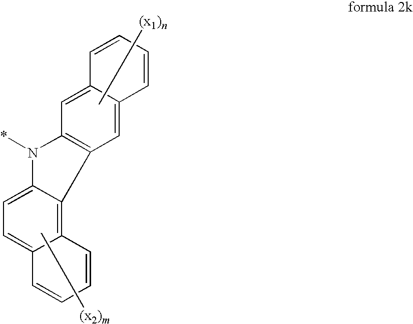

In Formulae 2a to 2k, * may be a binding site for Ar₁ or a benzophenanthrene ring.

In Formulae 2a to 2k, X₁ and X₂ may, for example, be each independently a C₁-C₁₀ alkyl group, a C₅-C₁₄ aryl group, or —Si(R₂₁)(R₂₂)(R₂₃), and particularly a methyl group, an ethyl group, a propyl group, a butyl group, a phenyl group, a naphthyl group, an anthryl group, a methylsilyl group, an ethylsilyl group, a dimethylsilyl group, a diethylsily group, a trimethylsilyl group (TMS), or triethylsilyl group. In addition, n and m are each independently an integer of 0-6.

Meanwhile, in Formula 2, a may be 0. If a is 1 or greater, the group of

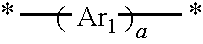

may be represented by one of Formulae 2a′ to 2g′ below, but is not limited thereto.

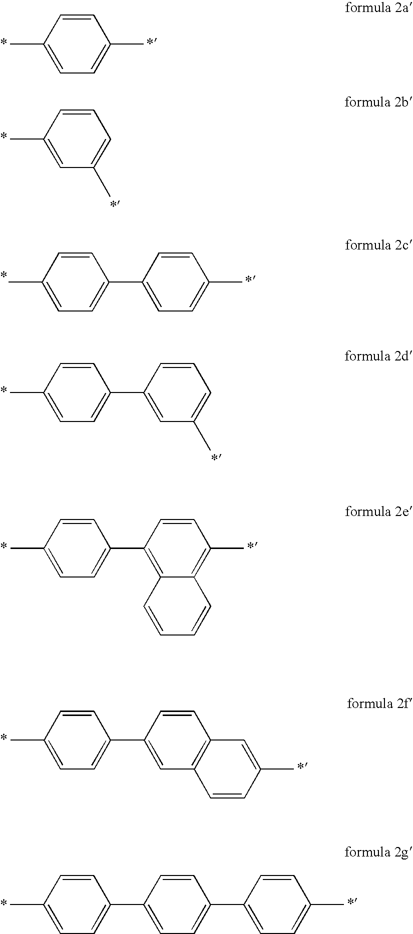

The fused aromatic compound represented by Formula 1 may be one of Compounds 1 to 12 below, but is not limited thereto.

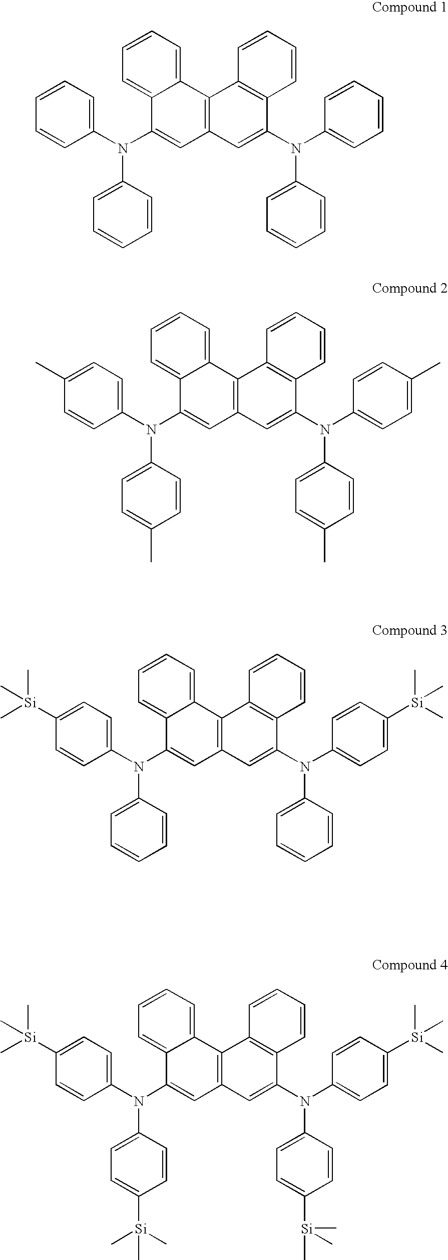

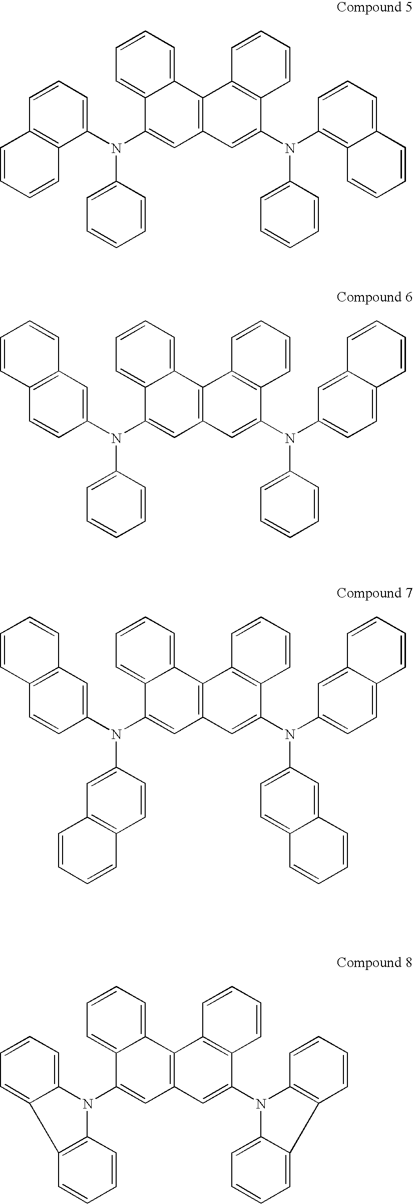

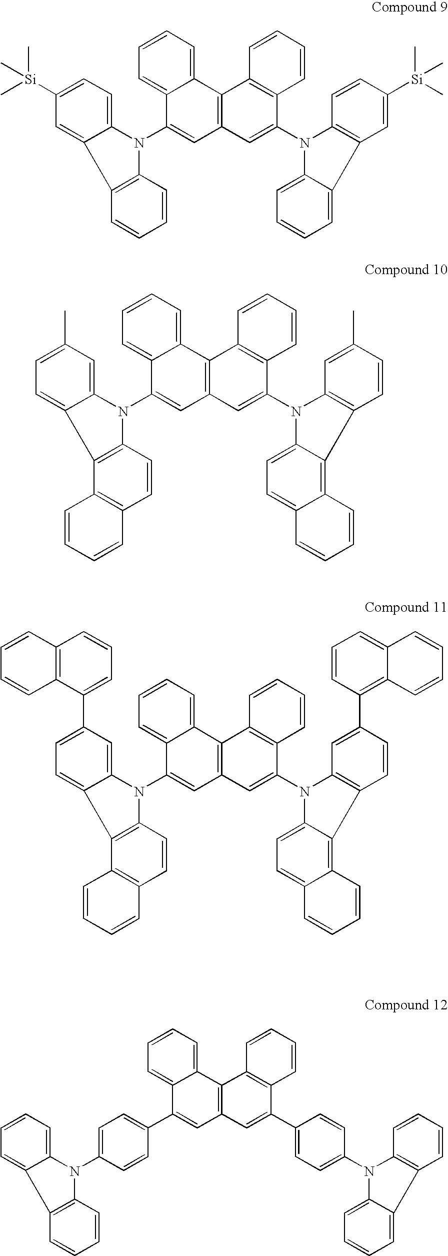

A fused aromatic compound according to another embodiment of the present invention may be represented by, for example, Formula 3 below.

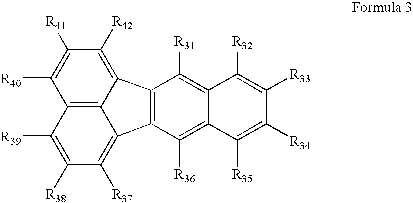

In Formula 3, R₃₁ to R₃₆ may, for example, be each independently a hydrogen atom, a halogen atom, a cyano group, a nitro group, a hydroxyl group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₅-C₆₀ cycloalkyl group, a substituted or unsubstituted C₅-C₆₀ cycloalkenyl group, a substituted or unsubstituted C₅-C₆₀ aryl group, a substituted or unsubstituted C₁-C₆₀ heteroaryl group, a group represented by Formula 4, or a group represented by Formula 5.

For example, R₃₁ to R₃₆ may be each independently a hydrogen atom, a halogen atom, a cyano group, a nitro group, a hydroxyl group, a substituted or unsubstituted C₁-C₁₀ alkyl group, a substituted or unsubstituted C₂-C₁₀ alkenyl group, a substituted or unsubstituted C₂-C₁₀ alkynyl group, a substituted or unsubstituted C₁-C₁₀ alkoxy group, a substituted or unsubstituted C₅-C₁₄ cycloalkyl group, a substituted or unsubstituted C₅-C₁₄ cycloalkenyl group, a substituted or unsubstituted C₅-C₁₄ aryl group, a substituted or unsubstituted C₁-C₁₄ heteroaryl group, the group represented by Formula 4, or the group represented by Formula 5. In more particular, R₃₁ to R₃₆ may be each independently a hydrogen atom, a halogen atom, a cyano group, a nitro group, a hydroxyl group, a C₁-C₁₀ alkyl group, a C₂-C₁₀ alkenyl group, a C₂-C₁₀ alkynyl group, a C₁-C₁₀ alkoxy group, a C₅-C₁₄ aryl group, the group represented by Formula 4 below, or the group represented by Formula 5 below, but are not limited thereto.

In Formula 3, R₃₇ to R₄₂ may, for example, be each independently a hydrogen atom or the group represented by Formula 5 below.

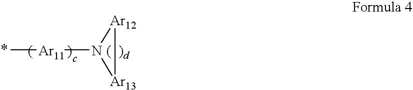

*-(Ar₁₄)ₓ-Q₁ Formula 5

In Formulae 4 and 5, Ar₁₁ and Ar₁₄ may, for example, be each independently a C₅-C₆₀ arylene group; or a C₅-C₆₀ arylene group substituted with at least one selected from the group consisting of a halogen atom, a cyano group, a nitro group, a hydroxyl group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₅-C₆₀ cycloalkyl group, a substituted or unsubstituted C₅-C₆₀ cycloalkenyl group, a substituted or unsubstituted C₅-C₆₀ aryl group, a substituted or unsubstituted C₁-C₆₀ heteroaryl group, and —Si(R₄₄)(R₄₅)(R₄₆).

For example, Ar₁₁ and Ar₁₄ may be each independently a C₅-C₁₄ arylene group; or a C₅-C₁₄ arylene group substituted with at least one selected from the group consisting of a halogen atom, a cyano group, a nitro group, a hydroxyl group, a substituted or unsubstituted C₁-C₁₀ alkyl group, a substituted or unsubstituted C₂-C₁₀ alkenyl group, a substituted or unsubstituted C₂-C₁₀ alkynyl group, a substituted or unsubstituted C₁-C₁₀ alkoxy group, a substituted or unsubstituted C₅-C₁₄ cycloalkyl group, a substituted or unsubstituted C₅-C₁₄ cycloalkenyl group, a substituted or unsubstituted C₅-C₁₄ aryl group, a substituted or unsubstituted C₁-C₁₄ heteroaryl group, and —Si(R₄₄)(R₄₅)(R₄₆). In more particular, Ar₁₁ and Ar₁₄ may be each independently a C₅-C₁₄ arylene group; or a C₅-C₁₄ arylene group substituted with at least one selected from the group consisting of a halogen atom, a cyano group, a nitro group, a hydroxyl group, a C₁-C₁₀ alkyl group, a C₂-C₁₀ alkenyl group, a C₂-C₁₀ alkynyl group, a C₁-C₁₀ alkoxy group, a C₅-C₁₄ aryl group, and —Si(R₄₄)(R₄₅)(R₄₆). For example, Ar₁₁ and Ar₁₄ may be each independently a C₅-C₁₄ arylene group; or a C₅-C₁₄ arylene group substituted with at least one C₁-C₁₀ alkyl group, a C₅-C₁₄ arylene group substituted with at least one C₅-C₁₄ aryl group, or a C₅-C₁₄ arylene group substituted with at least one —Si(R₄₄)(R₄₅)(R₄₆), but are not limited thereto.

In Formulae 4 and 5, Ar₁₂ and Ar₁₃ may be each independently a C₅-C₁₄ aryl group, a C₅-C₁₄ aryl group substituted with at least one C₁-C₁₀ alkyl group, a C₅-C₁₄ aryl group substituted with at least one C₅-C₁₄ aryl group, or a C₅-C₁₄ aryl group substituted with at least one —Si(R₄₄)(R₄₅)(R₄₆), but are not limited thereto.

In Formulae 4 and 5, Q₁ may be, for example, a hydrogen atom or —Si(R₄₇)(R₄₈)(R₄₉).

In this regard, R₄₄ to R₄₉ may be each independently a hydrogen atom, a C₁-C₁₀ alkyl group, or a substituted or unsubstituted C₅-C₁₄ aryl group, but are not limited thereto.

In Formula 4, c may be an integer of 1 to 6, and d may be 0 or 1. When d is 0, Ar₁₂ is not bonded to Ar₁₃ via a single bond, and when d is 1, Ar₁₂ is bonded to Ar₁₃ via a single bond.

In Formula 5, x may be an integer of 0 to 6.

In Formula 3, not all of R₃₁ to R₄₂ are hydrogen atoms.

Meanwhile, when all of R₃₇ to R₄₂ in Formula 3 are hydrogen atoms, R₃₁ to R₃₆ are each independently a hydrogen atom, the group represented by Formula 4, or the group represented by Formula 5. Here, at least one of R₃₁ to R₃₆ may be the group represented by Formula 4, or the group represented by Formula 5 in which Q₁ is —Si(R₄₇)(R₄₈)(R₄₉). That is, when all of R₃₇ to R₄₂ are hydrogen atoms, at least one of R₃₁ to R₃₆ is the group represented by Formula 4, or have a substituent represented by —Si(R₄₇)(R₄₈)(R₄₉). The —Si(R₄₇)(R₄₈)(R₄₉) group may make a HOMO(Highest Occupied Molecular Orbita)/LUMO(Lowest Unoccupied Molecular Orbital) level of the substituted compound deeper, and thus an OLED including the compound may have excellent performance.

In addition, in Formula 3, when all of R₃₁ to R₃₆ are hydrogen atoms, at least one of R₃₇ to R₄₂ may be the group represented by Formula 5 in which Q₁ is —Si(R₄₇)(R₄₈)(R₄₉). That is, when all of R₃₁ to R₃₆ are hydrogen atoms, at least one of R₃₇ to R₄₂ have —Si(R₄₇)(R₄₈)(R₄₉). The —Si(R₄₇)(R₄₈)(R₄₉) group may make a HOMO/LUMO level of the substituted compound deeper, and thus an OLED including the compound may have excellent performance.

For example, the compound of Formula 3 may be represented by Formula 3a below.

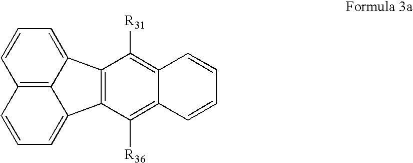

In Formula 3a, R₃₁ and R₃₆ may, for example, be each independently a hydrogen atom, provided that the group represented by Formula 4, or the group represented by Formula 5, wherein at least one of R₃₁ and R₃₆ may be the group represented by Formula 4 or the group represented by Formula 5 in which Q₁ is —Si(R₄₇)(R₄₈)(R₄₉).

In addition, the compound of Formula 3 may, for example, be represented by Formula 3b below.

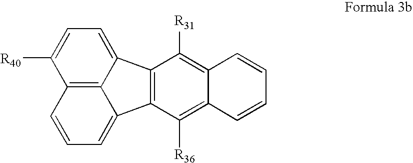

In Formula 3b, R₃₁, R₃₆ and R₄₀ are described above.

In Formula 4,

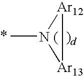

may be represented by one of Formulae 4a to 4k below, but is not limited thereto.

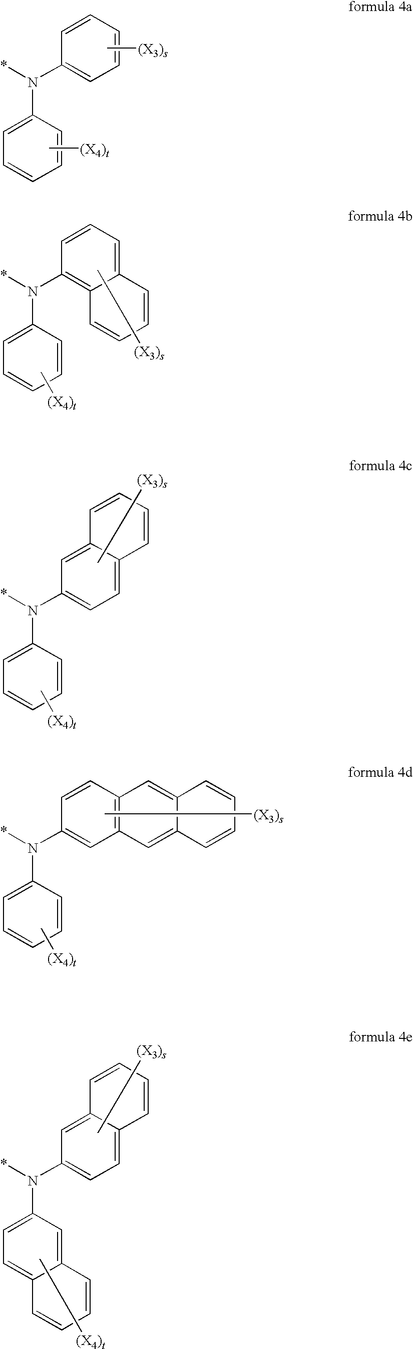

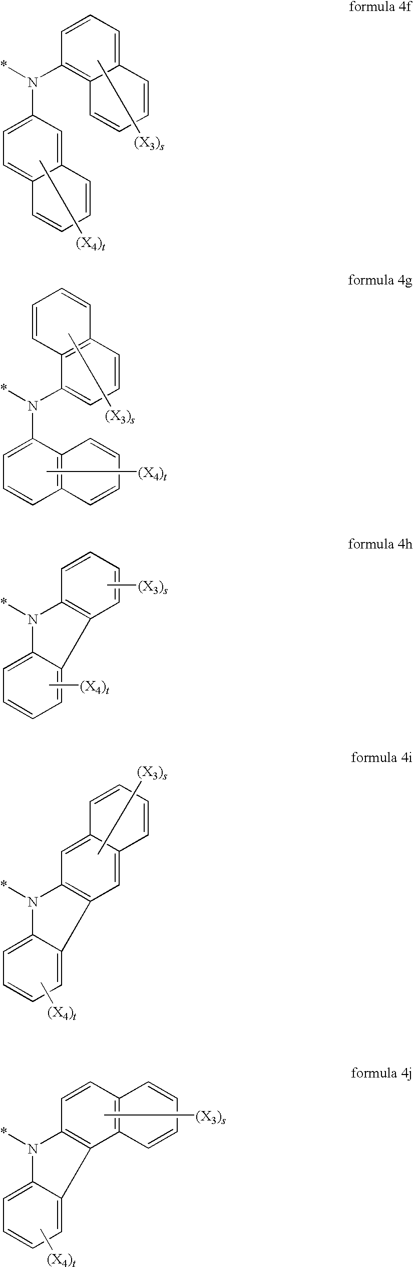

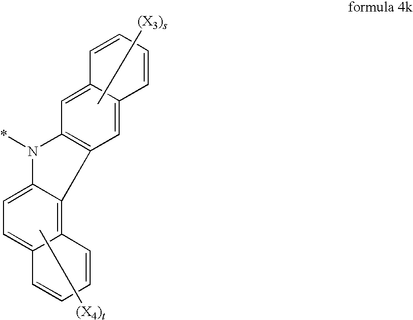

In Formulae 4a to 4k, * may be a binding site for a benzofluoranthene ring.

In Formulae 4a to 4k, X₃ and X₄ may, for example, be each independently a C₁-C₁₀ alkyl group, a C₅-C₁₄ aryl group, or —Si(R₄₄)(R₄₅)(R₄₆), and particularly, a methyl group, an ethyl group, a propyl group, a butyl group, a phenyl group, a naphthyl group, an anthryl group, a methylsilyl group, an ethylsilyl group, a dimethylsilyl group, a diethylsily group, a trimethylsilyl group, or a triethylsilyl group. In addition, s and t may be each independently an integer of 0 to 6.

In Formula 4, *Ar₁ₐ* may be represented by one of Formulae 4a′ to 4g′ below, but is not limited thereto.

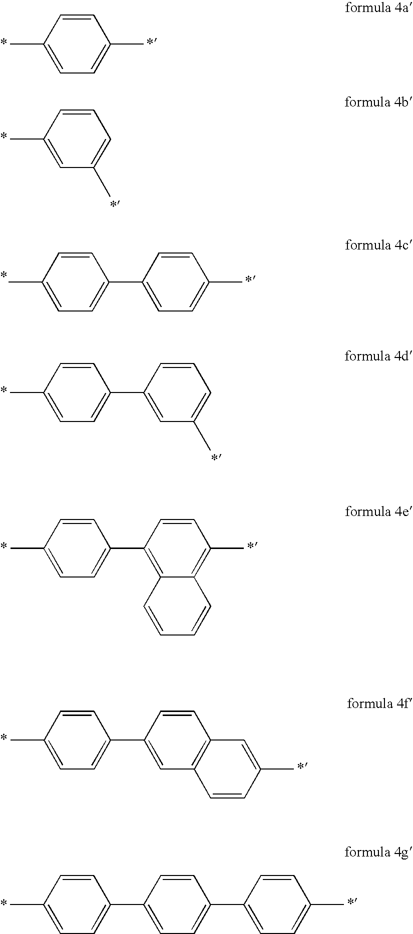

The group represented by Formula 5 may be represented by one of Formulae 5a to 5f below, but is not limited thereto.

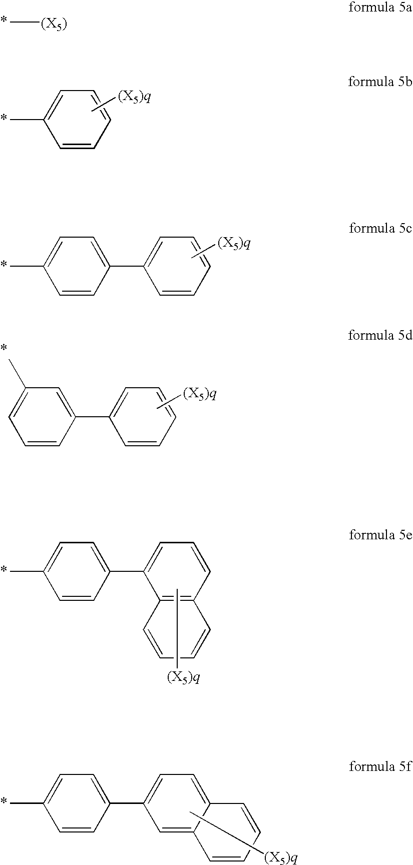

In Formulae 5a to 5f, X₅ may, for example, be a C₁-C₁₀ alkyl group, a C₅-C₁₄ aryl group, or —Si(R₄₇)(R₄₈)(R₄₉), and particularly a methyl group, an ethyl group, a propyl group, a butyl group, a phenyl group, a naphthyl group, an anthryl group, a methylsilyl group, an ethylsilyl group, a dimethylsilyl group, a diethylsily group, a trimethylsilyl group, or a triethylsilyl group. In addition, q may be an integer of 0 to 6.

The fused aromatic compound represented by Formula 3 may be one of Compounds 13 to 38 below, but is not limited thereto.

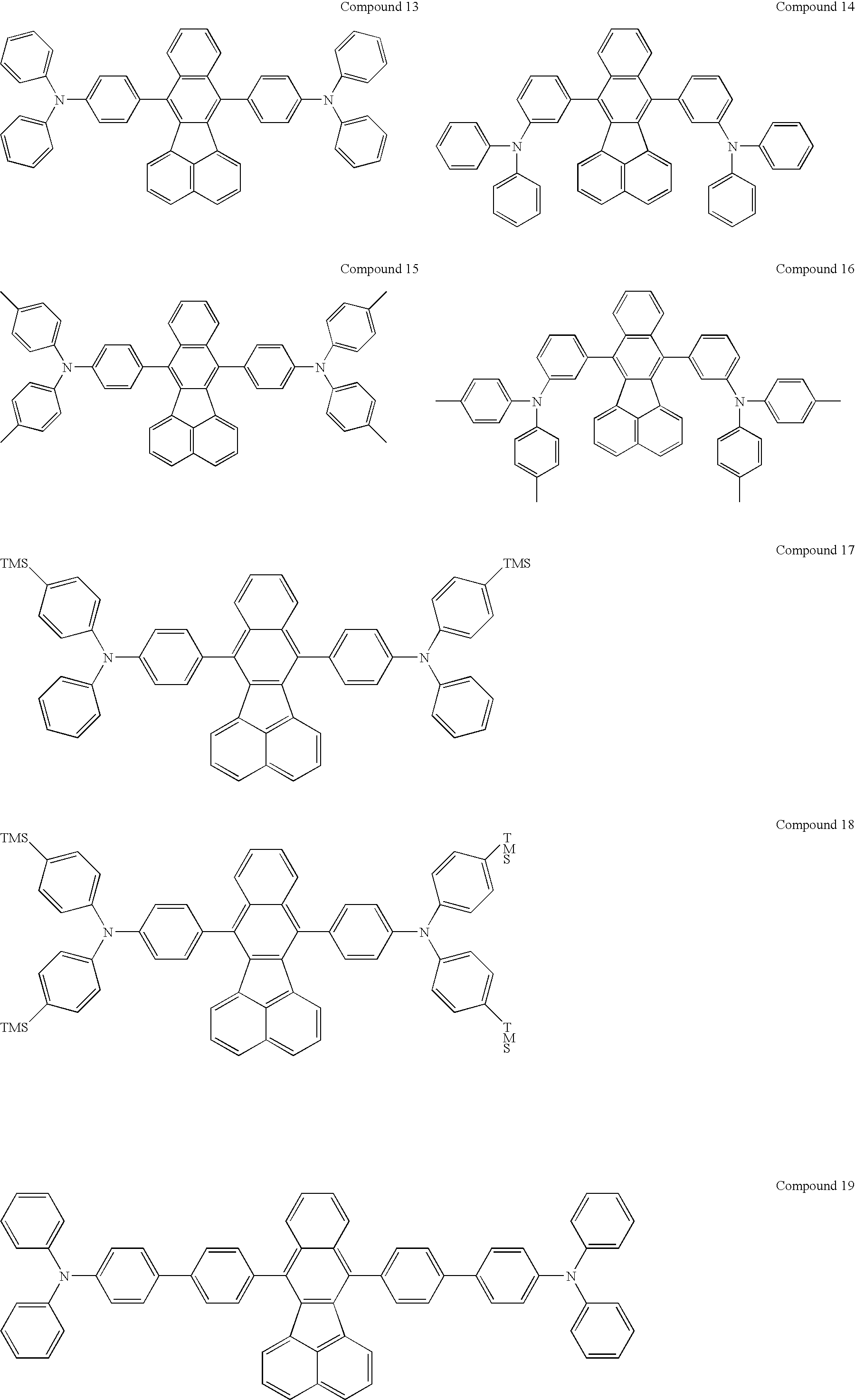

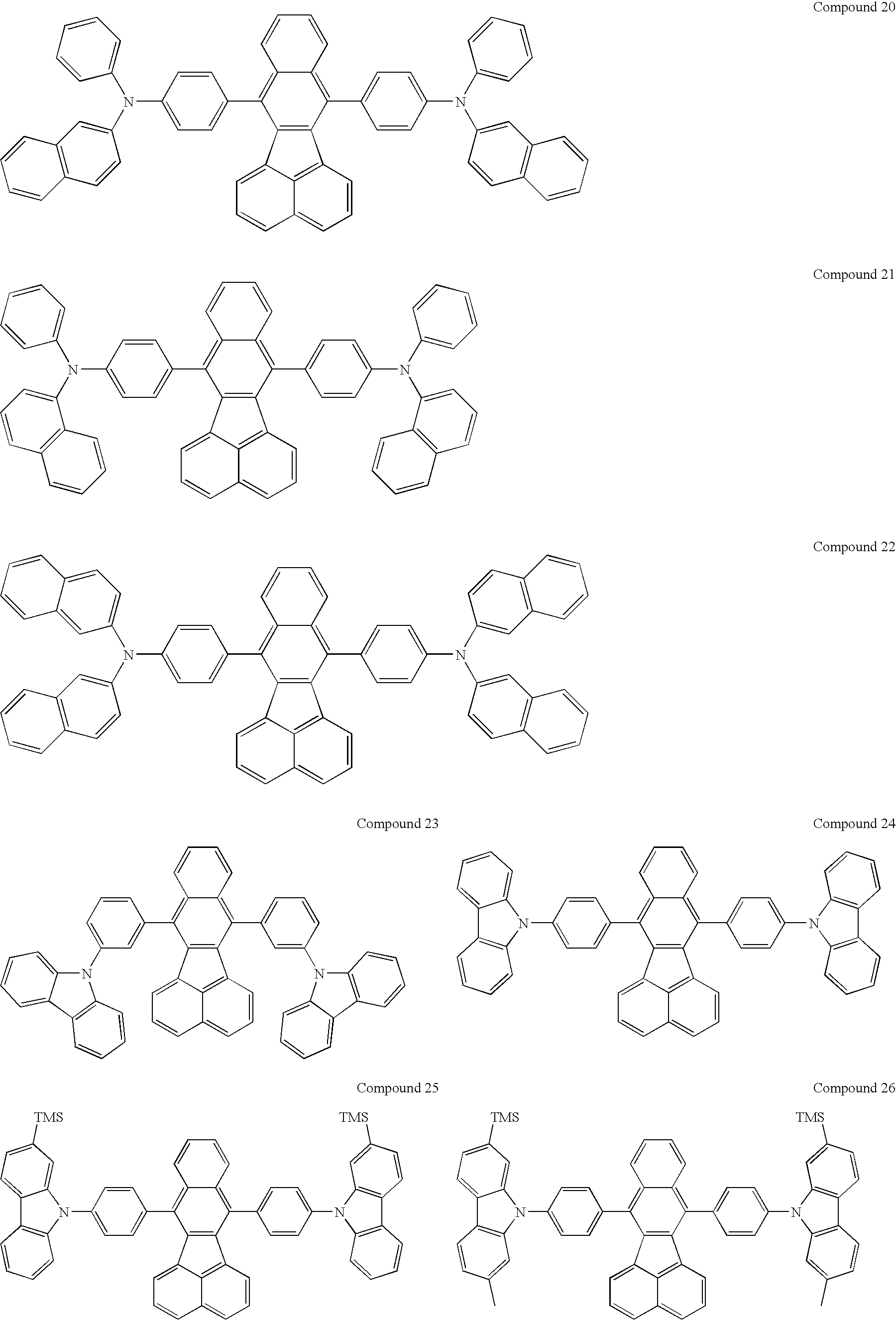

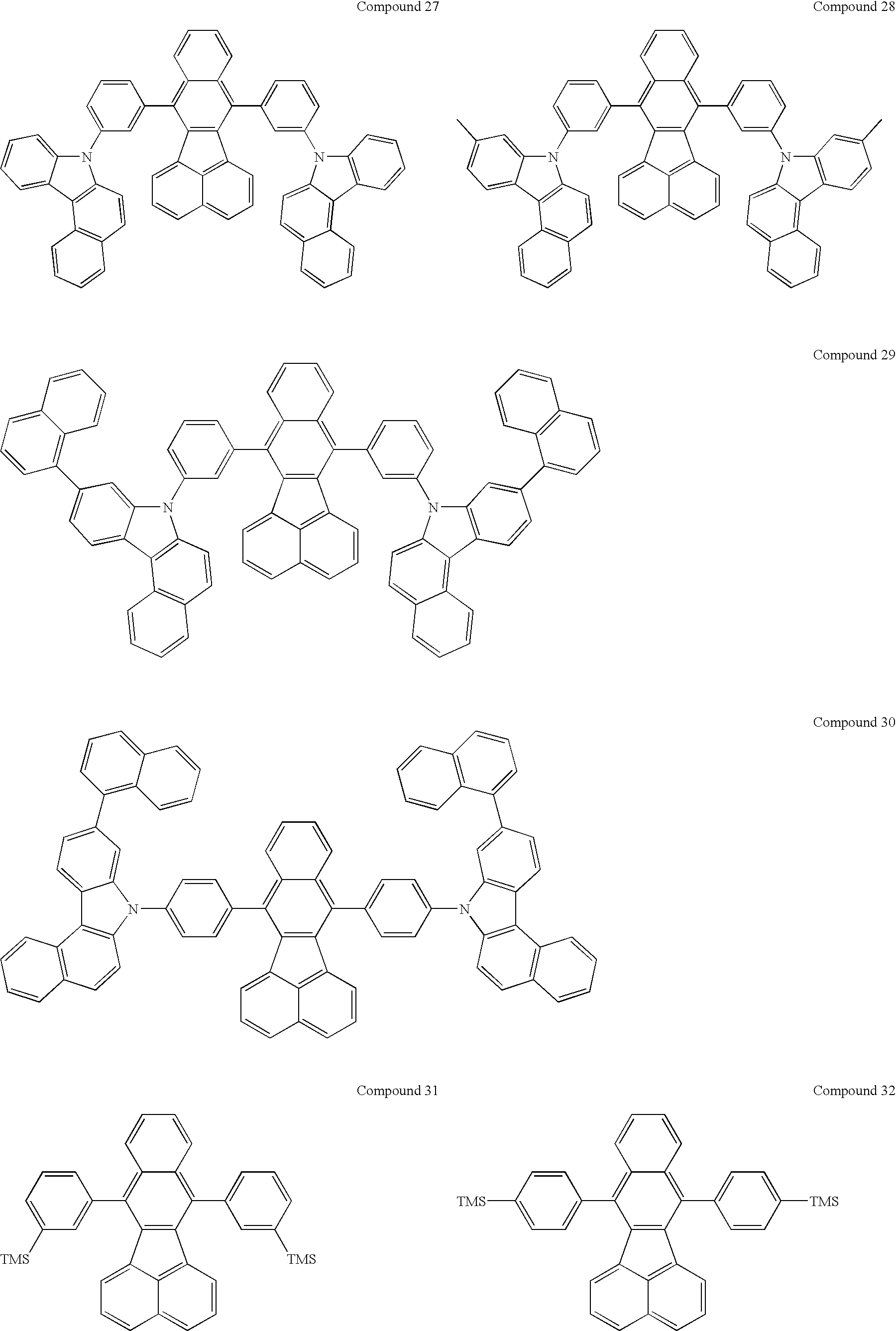

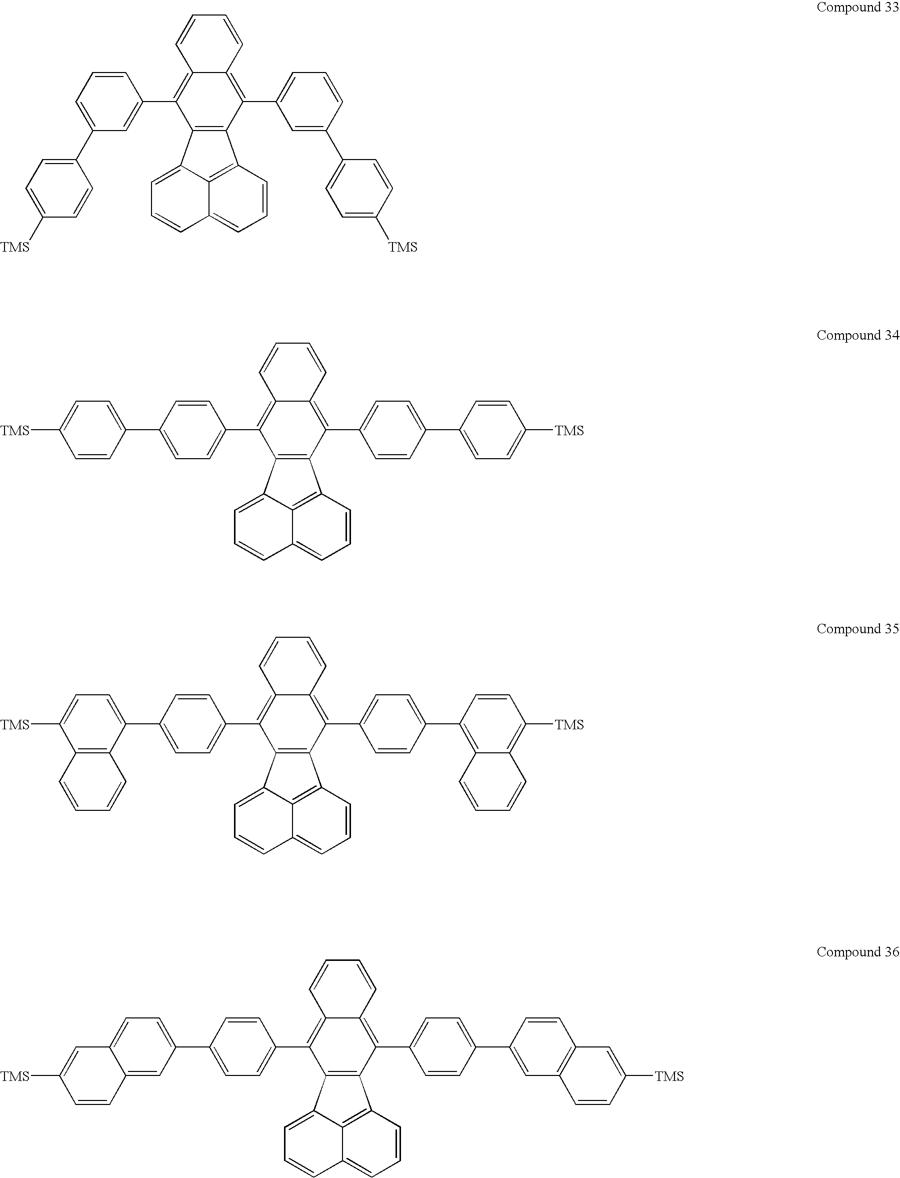

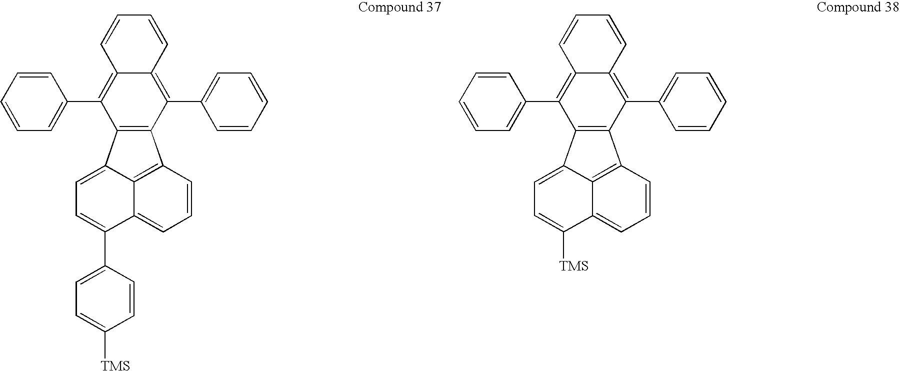

A fused aromatic compound according to another embodiment of the present invention may be represented by, for example, Formula 6 below.

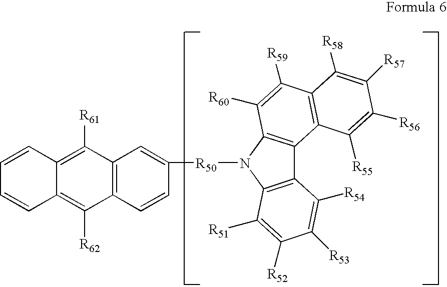

In Formula 6, one of R₅₀ to R₆₀ is bonded to an anthracene ring of Formula 6.

In Formula 6, R₅₀ to R₆₀ may, for example, be each independently a hydrogen atom, a halogen atom, a cyano group, a nitro group, a hydroxyl group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₅-C₆₀ cycloalkyl group, a substituted or unsubstituted C₅-C₆₀ cycloalkenyl group, a substituted or unsubstituted C₅-C₆₀ aryl group, a substituted or unsubstituted C₁-C₆₀ heteroaryl group, or —Si(R₆₅)(R₆₆)(R₆₇). In this regard, at least two adjacent groups among R₅₀ to R₆₀ may be bonded to each other to form a ring.

For example, R₅₀ to R₆₀ may be each independently a hydrogen atom, a halogen atom, a cyano group, a nitro group, a hydroxyl group, a substituted or unsubstituted C₁-C₁₀ alkyl group, a substituted or unsubstituted C₂-C₁₀ alkenyl group, a substituted or unsubstituted C₂-C₁₀ alkynyl group, a substituted or unsubstituted C₁-C₁₀ alkoxy group, a substituted or unsubstituted C₅-C₁₄ cycloalkyl group, a substituted or unsubstituted C₅-C₁₄ cycloalkenyl group, a substituted or unsubstituted C₅-C₁₄ aryl group, a substituted or unsubstituted C₁-C₁₄ heteroaryl group, or —Si(R₆₅)(R₆₆)(R₆₇). In more particular, R₅₀ to R₆₀ may be each independently a hydrogen atom, a C₁-C₁₀ alkyl group, or a C₅-C₁₄ aryl group, but are not limited thereto.

In this regard, R₆₅ to R₆₇ may be each independently a hydrogen atom, a C₁-C₁₀ alkyl group, a C₂-C₁₀ alkenyl group, or a C₅-C₁₄ aryl group, but are not limited thereto.

In Formula 6, R₆₁ and R₆₂ may be each independently a substituted or unsubstituted C₁-C₆₀ alkyl group or a phenyl group.

In particular, R₆₁ and R₆₂ may be each independently a C₁-C₁₀ alkyl group or a phenyl group, but are not limited thereto.

The fused aromatic compound of Formula 6 may be represented by Formula 6a or 6b.

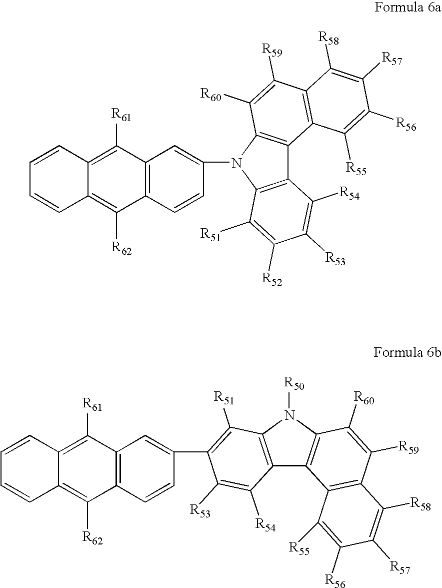

In Formula 6a and 6b, R₅₀ to R₆₂ are described above.

The fused aromatic compound represented by Formula 6 may be any one of Compounds 39 to 43, but is not limited thereto.

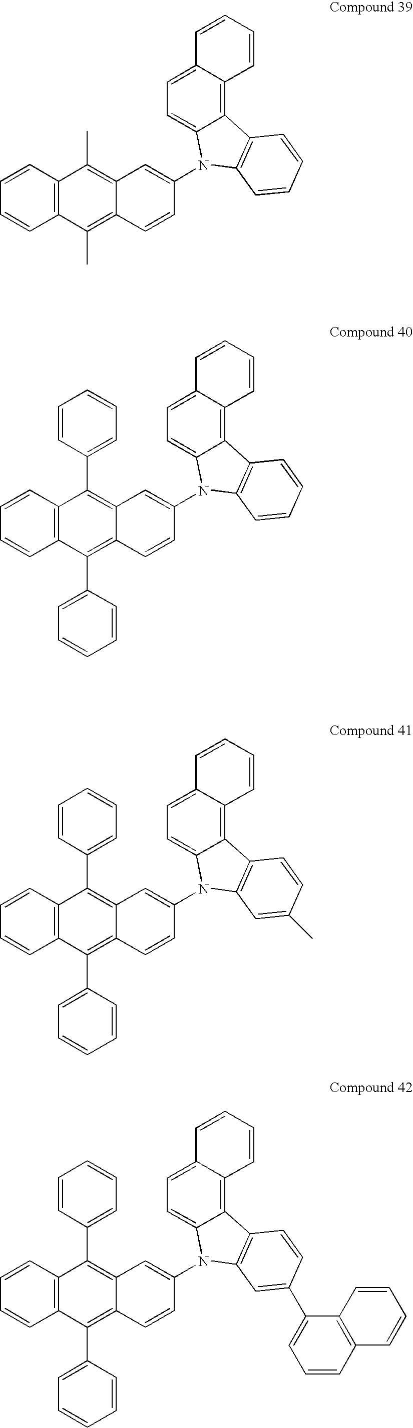

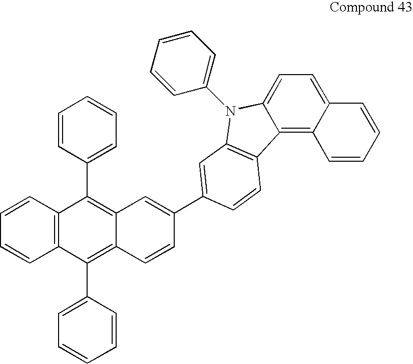

A fused aromatic compound according to another embodiment of the present invention may be represented by, for example, Formula 7 below.

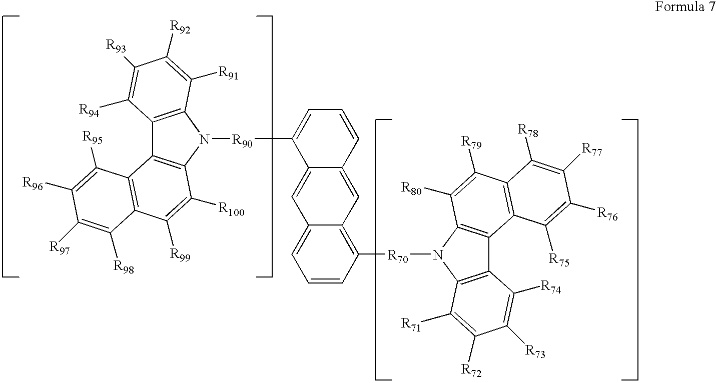

In Formula 7, one of R₇₀ to R₈₀ and one of R₉₀ to R₁₀₀ are bonded to an anthracene ring of Formula 7.

In Formula 7, R₇₀ to R₈₀ and R₉₀ to R₁₀₀ may, for example, be each independently a hydrogen atom, a halogen atom, a cyano group, a nitro group, a hydroxyl group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₅-C₆₀ cycloalkyl group, a substituted or unsubstituted C₅-C₆₀ cycloalkenyl group, a substituted or unsubstituted C₅-C₆₀ aryl group, a substituted or unsubstituted C₁-C₆₀ heteroaryl group, or —Si(R₁₀₁)(R₁₀₂)(R₁₀₃), wherein at least two adjacent groups among R₇₀ to R₈₀ and R₉₀ to R₁₀₀ may be bonded to each other to form a ring.

In particular, R₇₀ to R₈₀ and R₉₀ to R₁₀₀ may be each independently a hydrogen atom, a halogen atom, a cyano group, a nitro group, a hydroxyl group, a substituted or unsubstituted C₁-C₁₀ alkyl group, a substituted or unsubstituted C₂-C₁₀ alkenyl group, a substituted or unsubstituted C₂-C₁₀ alkynyl group, a substituted or unsubstituted C₁-C₁₀ alkoxy group, a substituted or unsubstituted C₅-C₁₄ cycloalkyl group, a substituted or unsubstituted C₅-C₁₄ cycloalkenyl group, a substituted or unsubstituted C₅-C₁₄ aryl group, a substituted or unsubstituted C₁-C₁₄ heteroaryl group, or —Si(R₁₀₁)(R₁₀₂)(R₁₀₃), and more particular, a hydrogen atom, a C₁-C₁₀ alkyl group, or a C₅-C₁₄ aryl group, but are not limited thereto.

In this regard, R₁₀₁ to R₁₀₃ may be each independently a hydrogen atom, a C₁-C₁₀ alkyl group, a C₂-C₁₀ alkenyl group, or a C₅-C₁₄ aryl group, but are not limited thereto.

The fused aromatic compound of Formula 7 may be represented by Formula 7a or 7b below.

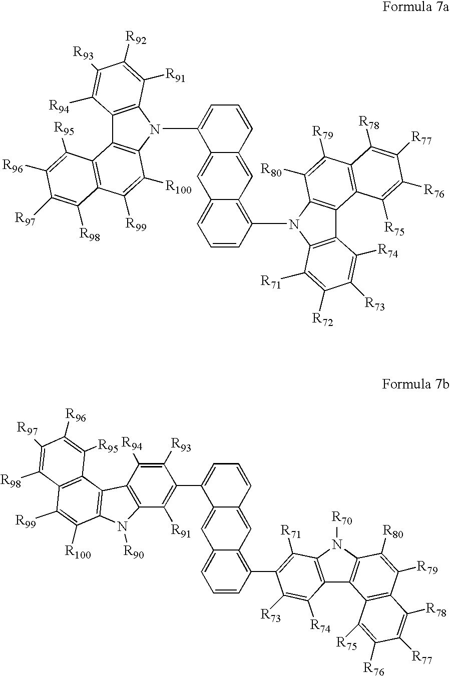

The fused aromatic compound represented by Formula 7 may be any one of Compounds 44 to 46, but is not limited thereto.

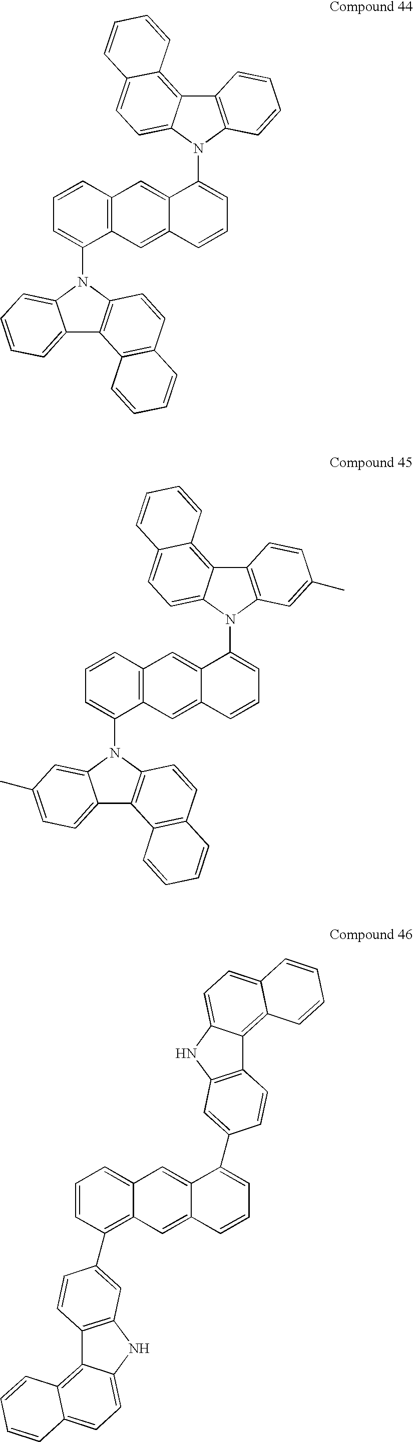

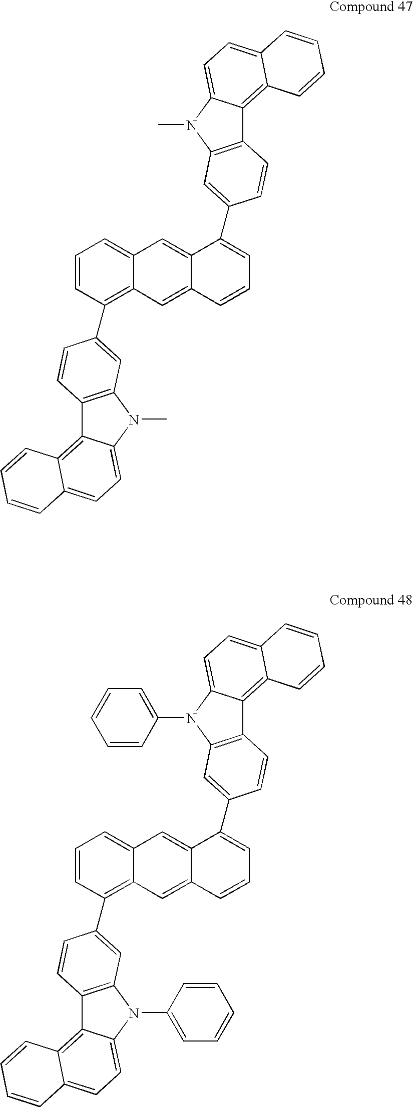

The unsubstituted C₁-C₆₀ alkyl group may, for example, be a linear or branched alkyl group, and examples of the unsubstituted C₁-C₆₀ alkyl group are methyl, ethyl, propyl, isobutyl, sec-butyl, pentyl, iso-amyl, hexyl, heptyl, octyl, nonanyl, and dodecyl. At least one hydrogen atom in the unsubstituted C₁-C₆₀ alkyl group may be substituted with a halogen atom, a hydroxy group, a nitro group, a cyano group, an amino group, an amidino group, hydrazine, hydrazone, carboxylic acid or a salt thereof, sulfonic acid or a salt thereof, phosphoric acid or a salt thereof, a C₁-C30 alkyl group, a C₁-C30 alkoxy group, a C₁-C30 alkenyl group, a C₁-C₃₀ alkynyl group, a C₆-C₃₀ aryl group, a C₁-C₃₀ heteroaryl group, or —Si(Q₂)(Q₃)(Q₄). In this regard, Q₂ to Q₄ may be each independently a hydrogen atom, a C₁-C₃₀ alkyl group, a C₁-C₃₀ haloalkyl group, a C₆-C₃₀ aryl group, a C₆-C₃₀ haloaryl group, or a C₂-C₃₀ heteroaryl group.

The unsubstituted C₂-C₆₀ alkenyl group indicates an alkyl group having a carbon-carbon double bond in the center or at a terminal of the alkyl group. Examples of the unsubstituted C₂-C₆₀ alkenyl group include but are not limited to ethenyl, propenyl, butenyl, pentenyl, hexenyl, heptenyl, and octenyl, but are not limited thereto. At least one hydrogen atom of the unsubstituted C₂-C₆₀ alkenyl group may be substituted with the substituents described with reference to the C₁-C₆₀ alkyl group.

The unsubstituted C₂-C₆₀ alkynyl group indicates an alkyl group having a carbon-carbon triple bond in the center or at a terminal of the alkyl group. Examples of the unsubstituted C₂-C₆₀ alkynyl group are acetylenyl and propylenyl, but are not limited thereto. At least one hydrogen atom of the unsubstituted C₂-C₆₀ alkynyl group may be substituted with the substituents described with reference to the C₁-C₆₀ alkyl group.

The unsubstituted C₁-C₆₀ alkoxy group indicates a group having —OA (A is the unsubstituted C₁-C₆₀ alkyl group described above). Examples of the unsubstituted C₁-C₆₀ alkoxy group are methoxy, ethoxy, propoxy, isopropoxy, butoxy, and pentoxy, but are not limited thereto. At least one hydrogen atom of the unsubstituted C₁-C₆₀ alkoxy group may be substituted with the substituents described with reference to the C₁-C₆₀ alkyl group.

The unsubstituted C₅-C₆₀ cycloalkyl group indicates a C₅-C₆₀ alkyl group having a ring. Examples of the unsubstituted C₅-C₆₀ cycloalkyl group are cyclopentyl, cyclohexyl, and cycloheptyl, but are not limited thereto. At least one hydrogen atom of the unsubstituted C₅-C₆₀ cycloalkyl group may be substituted with the substituents described with reference to the C₁-C₆₀ alkyl group.

The unsubstituted C₅-C₆₀ cycloalkenyl group indicates a C₅-C₆₀ alkenyl group having a ring. Examples of the unsubstituted C₅-C₆₀ cycloalkenyl group are cyclohexenyl and cycloheptenyl, but are not limited thereto. At least one hydrogen atom of the unsubstituted C₅-C₆₀ cycloalkenyl group may be substituted with the substituents described with reference to the C₁-C₆₀ alkyl group.

The unsubstituted C₅-C₆₀ aryl group indicates an aromatic carbocyclic system having 5 to 60 carbon atoms and at least one aromatic ring. If the C₅-C₆₀ aryl group has two or more aromatic rings, the rings may be bondeded to each other by a single bond or fused with each other. At least one hydrogen atom of the unsubstituted C₅-C₆₀ aryl group may be substituted with the substituents described with reference to the C₁-C₆₀ alkyl group.

Examples of the substituted or unsubstituted C₅-C₆₀ aryl group include but are not limited to a phenyl group, a C₁-C₁₀ alkylphenyl group (e.g., an ethylphenyl group), a halophenyl group (e.g., an o-, m- and p-fluorophenyl group, and a dichlorophenyl group), a cyanophenyl group, a dicyanophenyl group, a trifluoromethoxyphenyl group, a biphenyl group, a halo biphenyl group, a cyano biphenyl group, a C₁-C₁₀ biphenyl group, a C₁-C₁₀ alkoxy biphenyl group, an o-, m-, and p-tolyl group, an o-, m- and p-cumenyl group, a mesityl group, a phenoxyphenyl group, an (α,α-dimethylbenzene)phenyl group, a (N,N′-dimethyl)aminophenyl group, a (N,N′-diphenyl)aminophenyl group, a pentalenyl group, an indenyl group, a naphthyl group, a halonaphthyl group (e.g., a fluoronaphthyl group), a C₁-C₁₀ alkylnaphthyl group (e.g., a methylnaphthyl group), a C₁-C₁₀ alkoxynaphthyl group (e.g., a methoxynaphthyl group), a cyanonaphthyl group, an anthracenyl group, an azulenyl group, a heptalenyl group, an acenaphthylenyl group, a phenalenyl group, a fluorenyl group, an anthraquinolyl group, a methylanthryl group, a phenanthryl group, a triphenylene group, a pyrenyl group, a chrysenyl group, an ethyl-chrysenyl group, a picenyl group, a perylenyl group, a chloroperylenyl group, a pentaphenyl group, a pentacenyl group, a tetraphenylenyl group, a hexaphenyl group, a hexacenyl group, a rubicenyl group, a coroneryl group, a trinaphthylenyl group, a heptaphenyl group, a heptacenyl, a pyranthrenyl group, or an ovalenyl group.

The unsubstituted C₅-C₆₀ arylene group is a bivalent linking group having a structure similar to that of the aryl group. Examples of the unsubstituted C₅-C₆₀ arylene group are a phenylene group and a naphthylene group, but are not limited thereto. At least one hydrogen atom of the unsubstituted C₅-C₆₀ arylene group may be substituted with the substituents described with reference to the C₁-C₆₀ alkyl group.

The unsubstituted C₁-C₆₀ heteroaryl group is an aromatic ring system including carbon rings and at least one hetero atom selected from the group consisting of N, O, P or S, wherein at least two aromatic rings may be fused with each other or bonded by a single bond. At least one hydrogen atom of the unsubstituted C₁-C₆₀ heteroaryl group may be substituted with the substituents described with reference to the C₁-C₆₀ alkyl group.

Examples of the unsubstituted C₁-C₃₀ heteroaryl group include but are not limited to a pyrazolyl group, an imidazolyl group, an oxazolyl group, a thiazolyl group, a triazolyl group, a tetrazolyl group, an oxadiazolyl group, a pyridinyl group, a pyridazinyl group, a pyrimidinyl group, a triazinyl group, a carbazolyl group, an indolyl group, a quinolinyl group and an isoquinolinyl group. At least one hydrogen atom of the unsubstituted C₁-C₃₀ heteroaryl group may be substituted with the substituents described with reference to the C₁-C₆₀ alkyl group.

The fused aromatic compound represented by Formula 1, 3, 6, or 7 may be synthesized using a conventional organic synthesis method.

The fused aromatic compound represented by Formula 1, 3, 6, or 7 may be included in an organic layer of an OLED. In this regard, the organic layer may be an emission layer (EML). The fused aromatic compound represented by Formula 1, 3, 6, or 7 may also be included in an EML (for example, a dopant of the EML), a hole injection layer (HIL), and a hole transport layer (HTL), but the use of the fused aromatic compound represented by Formula 1, 3, 6, or 7 is not limited thereto.

The organic layer including the fused aromatic compound represented by Formula 1, 3, 6, or 7 may be prepared using various conventional methods, for example, vacuum deposition or solution coating such as spin coating, inkjet printing, screen printing, casting, langmuir blodgett (LB), or spray printing. In addition, a thermal transfer method may be used. According to the one embodiment of thermal transfer method, an organic layer including the fused aromatic compound represented by Formula 1, 3, 6, or 7 is formed on a donor film using vacuum deposition or solution coating, and the organic layer is thermal-transferred to a substrate including a first electrode, and the like. A stable organic layer may be formed using the fused aromatic compound represented by Formula 1, 3, 6, or 7 due to its beneficial solubility and thermal stability. Therefore, an OLED having a low driving voltage, high efficiency, and beneficial brightness may be manufactured.

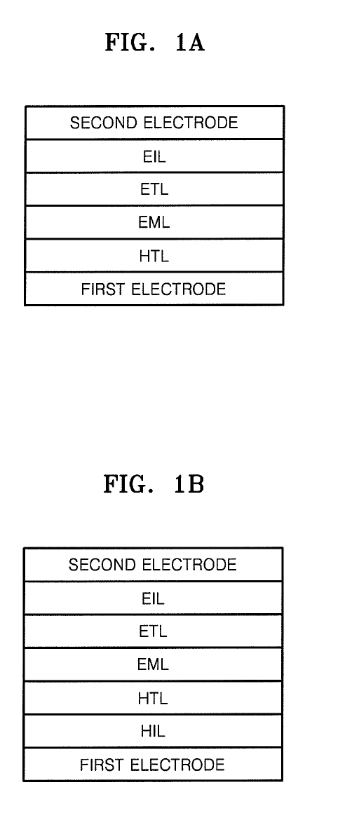

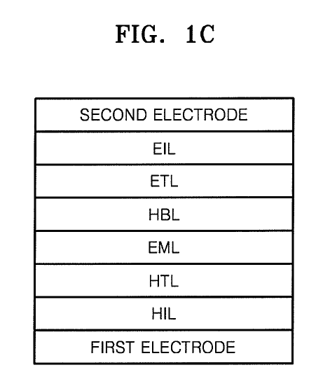

The OLED may include at least one layer selected from the group consisting of a hole injection layer (HIL), a hole transport layer (HTL), a hole blocking layer (HBL), an electron transport layer (ETL), and an electron injection layer (EIL) between a first electrode and a second electrode. In particular, FIGS. 1A to 1C schematically illustrate OLEDs according to embodiments of the present invention, but the structure is not limited thereto. The OLED of FIG. 1A has a first electrode/HTL/EML/ETL/EIL/second electrode structure, the OLED of FIG. 1B has a first electrode/HIL/HTL/EML/ETL/EIL/second electrode structure, and the OLED of FIG. 1C has a first electrode/HIL/HTL/EML/HBL/ETL/EIL/second electrode structure.

Hereinafter, a method of manufacturing an OLED according to an exemplary embodiment of the present invention will be described with reference to the OLED illustrated in FIG. 1C.

First, a substrate is prepared. The substrate, which may be any substrate that is used in conventional OLEDs, may be, for example, a glass substrate or a transparent plastic substrate that has beneficial mechanical strength, thermal stability, transparency, and surface smoothness, can be readily treated, and is waterproof.

Then, a first electrode is formed by, for example, depositing or sputtering a high work-function material on the substrate. The first electrode may be an anode, as a hole injection electrode on which a HIL is formed. The first electrode may be formed of, for example, ITO (Indium Tin Oxide), IZO (Indium Zinc Oxide), SnO₂, ZnO, or any transparent material which has high conductivity.

Then, a HIL may be formed on the first electrode by, for example, vacuum deposition, spin coating, casting, LB, or the like.

When the HIL is formed by vacuum deposition, vacuum deposition conditions may vary according to the compound that is used to form the HIL, and the desired structure and thermal properties of the HIL to be formed. In general, however, the vacuum deposition may be performed, for example, at a deposition temperature of about 100° C. to about 500° C., under a pressure of about 10⁻⁸ torr to about 10⁻³ torr, and at a deposition speed of about 0.01 to about 100 Å/sec.

When the HIL is formed by spin coating, coating conditions may vary according to a compound that is used to form the HIL, and the desired structure and thermal properties of the HIL to be formed. In general, however, the coating speed may be, for example, about 2000 rpm to about 5000 rpm, and a temperature for heat treatment, which is performed to remove a solvent after coating, may be about 80° C. to about 200° C.

The HIL may be formed of the fused aromatic compound represented by formula 1, 3, 6, or 7. Alternatively, the HIL may be formed of a conventional hole injection material, for example, a phthalocyanine compound such as copperphthalocyanine, 4,4′,4″-tris(3-methylphenylphenylamino)triphenylamine (m-MTDATA), N,N′-di(1-naphthyl)-N,N′-diphenylbenzidine) (NPB), TDATA, 2T-NATA, polyaniline/dodecylbenzenesulfonic acid (Pani/DBSA), poly(3,4-ethylenedioxythiophene)/poly(4-styrenesulfonate) (PEDOT/PSS), polyaniline/camphor sulfonicacid (Pani/CSA), or polyaniline/poly(4-styrenesulfonate) (PANI/PSS) without limitation.

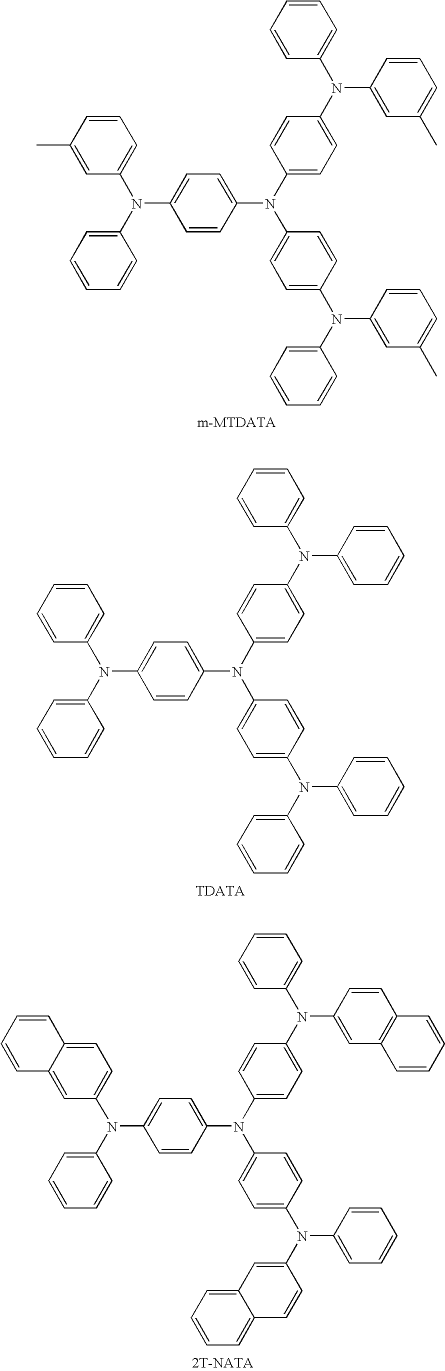

The thickness of the HIL may be, for example, about 100 Å to about 10000 Å, and for example, about 100 Å to about 1000 Å. If the thickness of the HIL is within the range described above, excellent hole injecting properties may be obtained without substantial increase in driving voltage.

Then, a HTL may be formed on the HIL using, for example, vacuum deposition, spin coating, casting, LB, or the like. When the HTL is formed by vacuum deposition or spin coating, the conditions for deposition and coating are similar to those for the formation of the HIL, although conditions for the deposition and coating may vary according to the material that is used to form the HTL.

The HTL may be formed of the fused aromatic compound represented by, for example, formula 1, 3, 6, or 7. Alternatively, the HTL may be formed of a conventional hole transporting material, for example, a carbazole derivative such as N-phenylcarbazole and polyvinylcarbazole; a typical amine derivative having an aromatic condensation ring such as N,N′-bis(3-methylphenyl)-N,N′-diphenyl-[1,1-biphenyl]-4,4′-diamine (TPD), N,N′-di(naphthalene-1-yl)-N,N′-diphenyl benzydine (α-NPD); a triphenylamine based material such as 4,4′,4″-tris(N-carbazolyl)triphenylamine) (TCTA), or the like. The TCTA may also inhibit exitons from being distributed from the EML in addition to hole transporting holes.

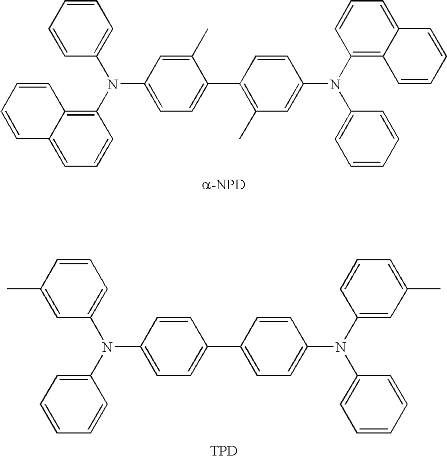

The thickness of the HTL may be about 50 Å to about 1000 Å, and for example, about 100 Å to about 800 Å. If the thickness of the HTL is within the range described above, beneficial hole transporting properties may be obtained without substantial increase in driving voltage.

Then, an EML may be formed on the HTL by, for example, vacuum deposition, spin coating, casting, LB, or the like. When the EML is formed by vacuum deposition or spin coating, the conditions for deposition and coating are similar to those for the formation of the HIL, although the conditions for deposition and coating may vary according to the material that is used to form the EML.

The EML may be formed of the fused aromatic compound represented by, for example, Formula 1, 3, 6, or 7. In this regard, the fused aromatic compound of Formula 1, 3, 6, or 7 may be used as a dopant and used with an appropriate host material. Also, a conventional dopant material may further be used. In addition, the fused aromatic compound of Formula 1, 3, 6, or 7 may be used as a host. Meanwhile, the fused aromatic compound of Formula 1, 3, 6, or 7 may be used by itself. The host material may be Aluminum this(8-hydroxyquinoline) (Alq₃), 4,4′-N,N′-dicarbazole-biphenyl (CBP), poly(n-binylcarbazole (PVK), 9,10-di(naphthalene-2-yl)anthracene (ADN), TCTA, 1,3,5-tris(N-phenylbenzimidazole-2-yl)benzene) (TPBI), 3-tert-butyl-9,10-di(naphth-2-yl)anthracene (TBADN), E3, or the like, but is not limited thereto.

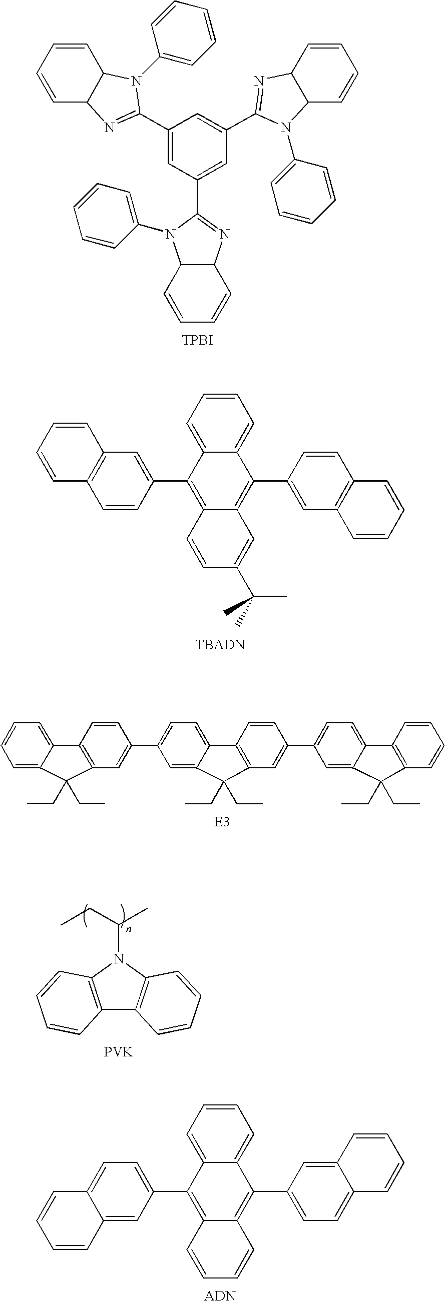

Meanwhile, a conventional red dopant may be PtOEP, Ir(piq)₃, Btp₂Ir(acac), 4-(dicyanomethylene)-2-t-butyl-6(1,1,7,7-tetramethyljulolidyl-9-enyl)-4H-pyran (DCJTB), or the like, but is not limited thereto.

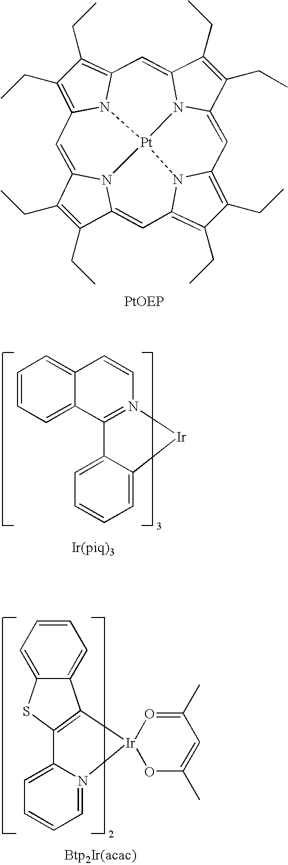

In addition, a conventional green dopant may be Ir(ppy)₃ (ppy=phenylpyridine), Ir(ppy)₂(acac), Ir(mpyp)₃, 10-(2-benzothiazolyl)-1,1,7,7,-tetramethyl-2,3,6,7-tetrahydro-1H,5H,11H-[1]benzo pyrano[6,7,8-ij]-quinolizin-11-one (C545T), or the like, but is not limited thereto.

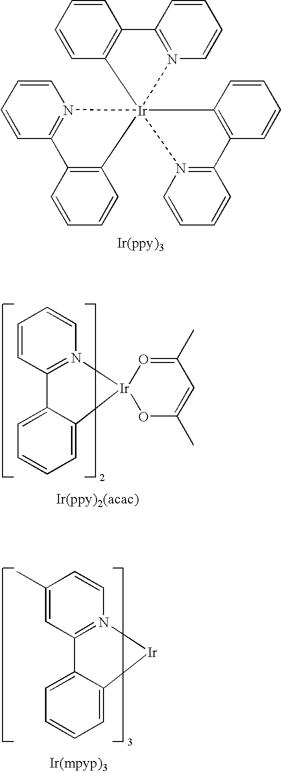

Meanwhile, a conventional blue dopant may be F₂Irpic, (F₂ppy)₂Ir(tmd), Ir(dfppz)₃, ter-fluorene, 4,4′-bis(4-diphenylaminostyryl)biphenyl (DPAVBi), 2,5,8,11-tetra-tert-butyl perylene (TBP), or the like, but is not limited thereto.

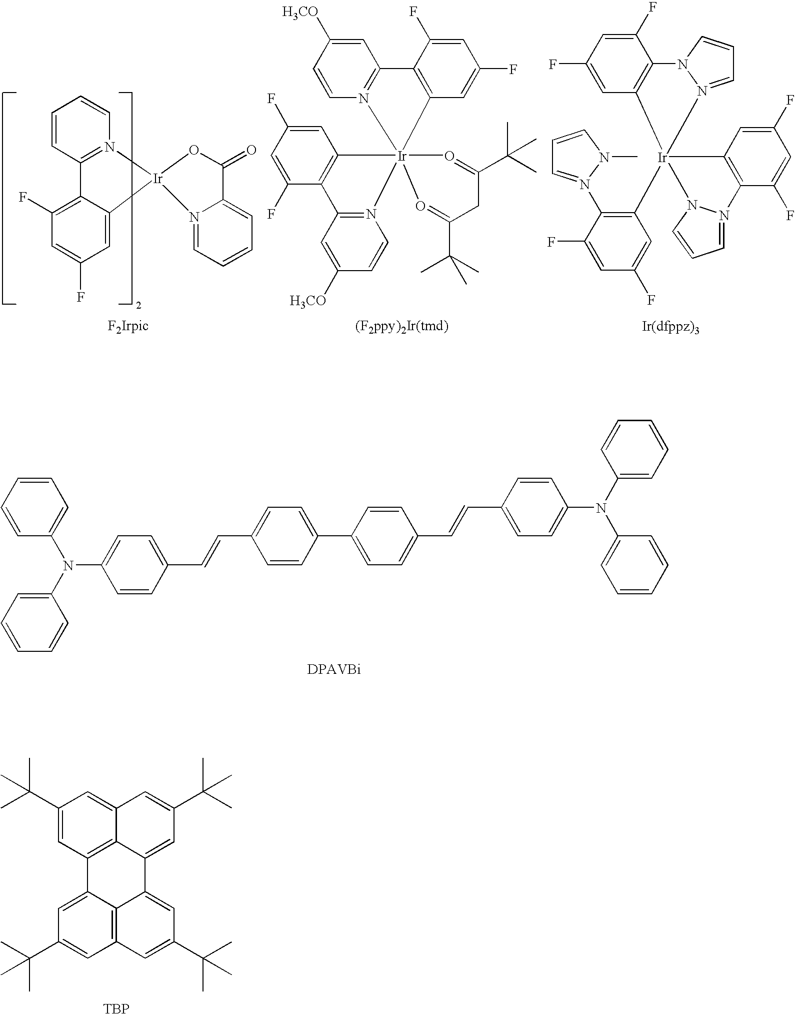

When the dopant is used together with the host, the concentration of the dopant is not limited, but may be about 0.01 to about 15 parts by weight based on 100 parts by weight of the host.

The thickness of the EML may be about 100 Å to about 1000 Å, and for example, about 200 Å to about 600 Å. if the thickness of the EML is within the range described above, beneficial light emitting properties may be obtained without substantial increase in driving voltage.

A HBL may be formed between the HTL and the EML using, for example, vacuum deposition, spin coating, casting, LB, or the like, to prevent diffusion of triplet excitons or holes into an ETL when the phosphorescent dopant is used to form the EML. When the HBL is formed by vacuum deposition or spin coating, the conditions for deposition and coating are similar to those for the formation of the HIL, although the conditions for deposition and coating may vary according to the material that is used to form the HBL. The HBL may be formed of a conventional material, for example, an oxadiazole derivative, a triazole derivative, or a phenanthroline derivative.

The thickness of the HBL may be in the range of, for example, about 50 Å to about 1000 Å, and for example, about 100 Å to about 300 Å. If the thickness of the HBL is within the range described above, beneficial hole blocking properties may be obtained without substantial increase in driving voltage.

Then, an ETL may be formed by, for example, vacuum deposition, spin coating, casting, or the like. When the ETL is formed by vacuum deposition or spin coating, the conditions for deposition and coating are, in general, similar to those for the formation of the HIL, although the conditions for the deposition and coating conditions may vary according to the material that is used to form the ETL. The ETL may be formed of a conventional material that stabily transports electrons injected from an electron injecting electrode (cathode), for example, a quinoline derivative, for example, Alq₃, TAZ, Balq or the like.

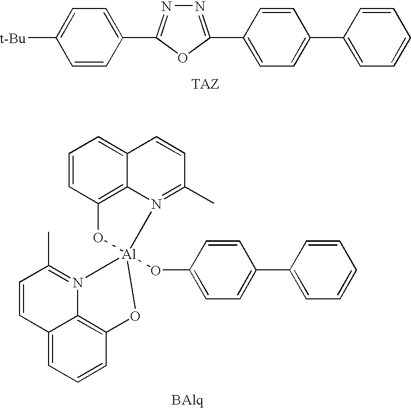

The thickness of the ETL may be, for example, about 100 Å to about 1000 Å, and for example, about 150 Å to about 500 Å. If the thickness of the ETL is within the range described above, excellent electron transporting properties may be obtained without substantial increase in driving voltage.

Then, an EIL, which is formed of a material allowing relatively easy injection of electrons from a cathode, may be formed on the ETL. The material that is used to form the EIL is not limited.

The EIL may be formed of, for example, lithium fluoride (LiF), sodium chloride (NaCl), cesium fluoride (CsF), lithium oxide (Li₂O), barium oxide (BaO), or the like, which is known in the art. Conditions for the deposition of the EIL are, in general, similar to conditions for the formation of the HIL, although they may vary according to the material that is used to form the EIL.

The thickness of the EIL may be, for example, about 1 Å to about 100 Å, and for example, about 5 Å to about 50 Å. If the thickness of the EIL is within the range described above, beneficial electron injecting properties may be obtained without substantial increase in driving voltage.

Finally, a second electrode may be formed on the EIL by, for example, vacuum deposition, sputtering, or the like. The second electrode may be used as a cathode. The second electrode may be formed of, for example, a low work-function metal, an alloy, an electrically conductive compound, or a combination of these. For example, the second electrode may be formed of lithium (Li), magnesium (Mg), aluminum (Al), aluminum-Lithium (Al—Li), calcium (Ca), magnesium-indium (Mg—In), magnesium-silver (Mg—Ag), or the like. Alternatively, a transparent cathode formed of, for example, indium tin oxide (ITO) or indium zinc oxide (IZO) may be used to produce a top emission type light emitting device.

A method of manufacturing an OLED according to one embodiment of the present invention may include, for example, forming a first electrode; forming an organic layer including the fused aromatic compound represented by, for example, Formula 1, 3, 6, or 7 on the first electrode; and forming a second electrode on the organic layer. The organic layer may be an EML. Meanwhile, the method may further include, for example, forming at least one layer selected from the group consisting of a HIL, a HTL, an EML, a HBL, an ETL and an EIL.

The organic layer including the fused aromatic compound represented by, for example, Formula 1, 3, 6, or 7 may be formed using, for example, vacuum deposition or solution coating such as spin coating, inkjet printing, screen printing, casting, LB, or spray printing. In addition, a thermal transfer method may be used. For example, according to one embodiment of the thermal transfer method, the organic layer including the fused aromatic compound represented by Formula 1, 3, 6, or 7 is formed on a donor film using vacuum deposition or solution coating, and the organic layer is thermal-transferred to a substrate including a first electrode, etc.

Hereinafter, the present invention will be described in more detail with reference to examples. The following examples are for illustrative purposes only and are not intended to limit the scope of the present invention.

### EXAMPLES

### Synthesis Example 1

Compound 2 was synthesized through Reaction Scheme 1 below:

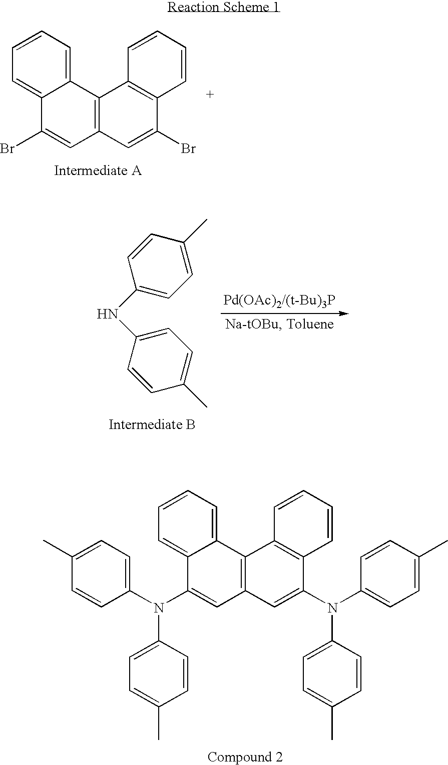

About 1.4 g (about 3.6 mmol) of Intermediate A prepared in Synthesis Example 1 (Intermediate A was synthesized with reference to Tetrahedron Letters 48 (2007) 6814-6816 of Jan Storch, etc.), about 1.4 g (about 7.2 mmole) of Intermediate B (di-p-tolylamine), about 40 mg (about 0.18 mol) of Pd(OAc)₂, about 109 mg (about 0.54 mmol) of P(t-Bu)₃, and about 1.0 g (about 10.9 mmol) of sodium t-butoxide were refluxed in about 15 ml of toluene in a nitrogen atmosphere for about 12 hours. When the reaction was terminated, the solvent was evaporated and removed. The resultant was washed respectively with about 500 ml of ethylacetate and about 500 ml of water. Then an organic layer was collected and dried using anhydrous magnesium sulfate. Then the resultant was isolated by silica chromatography to obtain about 1.6 g of Compound 2 (yield: about 71%).

¹H-NMR (CDCl₃, 300 MHz, ppm):9.0 (d, 2H), 8.2 (d, 2H), 7.6 (t, 2H), 7.4 (t, 2H), 7.3 (s, 2H), 7.0-6.9 (m, 16H), 2.3 (s, 12H).

Thermal analysis for Compound 2 was performed using thermo gravimetric analysis (TGA) and differential scanning calorimetry (DSC) under the following conditions: N₂ atmosphere, temperature of room temperature to about 600° C. (about 10° C./min)-TGA, and of room temperature to about 400° C.-DSC, Pan Type: Pt Pan in disposable Al Pan (TGA) and disposable Al pan (DSC). As a result, Tg of Compound 2 was about 140° C., Tm was about 249° C., and Td was about 417° C.

Meanwhile, a LUMO of Compound 2 was about −2.86 eV, a HOMO was about −5.70 eV, and an energy gap was about 2.84 eV (measured).

### Synthesis Example 2

Compound 15 was synthesized through Reaction Scheme 2 below:

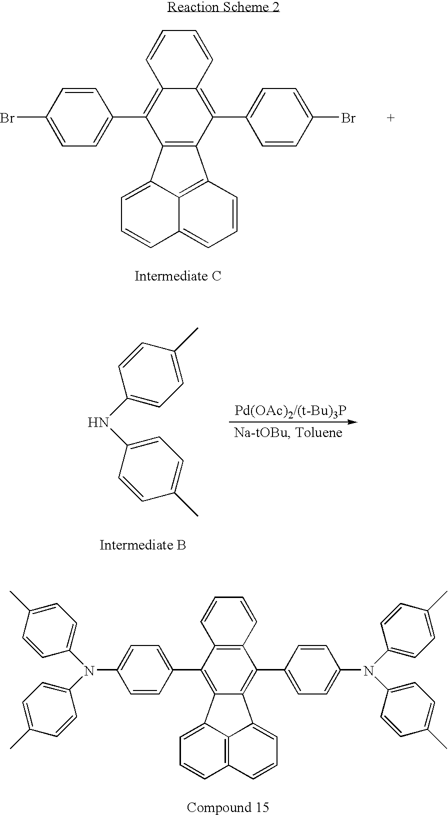

2.0 g (3.6 mmol) of Intermediate C prepared in Reaction Scheme 2 (Intermediate C was synthesized with reference to Tetrahedron Letters 38 (1997) 741-744 of Mark D. Clayton and Peter W. Rabideau), about 1.4 g (about 7.2 mmole) of Intermediate B, about 40 mg (about 0.18 mol) of Pd(OAc)₂, about 108 mg (about 0.54 mmol) of P(t-Bu)₃, and about 1.0 g (about 10.9 mmol) of sodium t-butoxide were refluxed in 14 ml of toluene in a nitrogen atmosphere for about 12 hours. When the reaction was terminated, the solvent was evaporated and removed. The resultant was washed respectively with about 500 ml of ethylacetate and about 500 ml of water. Then, an organic layer was collected and dried using anhydrous magnesium sulfate. Then the resultant was isolated by silica chromatography to obtain about 2.3 g of Compound 15 (yield: about 81%).

¹H-NMR (CDCl₃, 300 MHz, ppm):7.7-6.8 (m, 34H), 2.3 (s, 12H).

Thermal analysis for Compound 15 was performed using TGA and DSC under the following conditions: N₂ atmosphere, temperature of room temperature to 600° C. (about 10° C./min)-TGA, and of room temperature to about 400° C.-DSC, Pan Type: Pt Pan in disposable Al Pan (TGA) and disposable Al pan (DSC). As a result, Tg of Compound 15 was about 147° C., Tm was about 288° C., and Td was about 483° C.

Meanwhile, a LUMO of Compound 15 was about −2.9 eV, a HOMO was about −5.71 eV, and an energy gap was about 2.81 eV (measured).

### Synthesis Example 3

Compound 33 was synthesized through Reaction Scheme 3 below:

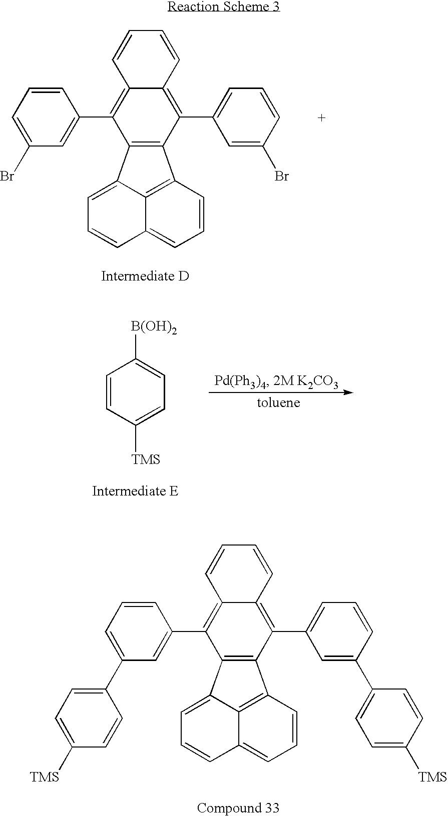

about 2.0 g (about 3.6 mmol) of Intermediate D (Intermediate D was synthesized with reference to Tetrahedron Letters 38 (1997) 741-744 by Mark D. Clayton and Peter W. Rabideau), about 1.5 g (about 7.9 mmol) of Intermediate E, about 208 mg (about 0.18 mmol) of tetrakis(triphenylphosphine)palladium (Pd(PPh₃)₄, and about 8 ml (about 16 mmol) of 2M potassium carbonate (K₂CO₃) solution were dissolved in about 10 ml of toluene and about 15 ml of tetrahydrofuran (THF), and the solution was refluxed for about 24 hours. When the reaction was terminated, the solvent was evaporated and removed. The resultant was washed respectively with about 500 ml of ethylacetate and about 500 ml of water. Then an organic layer was collected and dried using anhydrous magnesium sulfate. Then the resultant was isolated by silica chromatography to obtain about 1.1 g of Compound 33 (yield: about 44%).

¹H-NMR (CDCl₃, 300 MHz, ppm):7.9-7.2 (m, 24H), 6.7 (d, 2H), 0.3 (s, 18H).

Thermal analysis for Compound 33 was performed using DSC under the following conditions: N₂ atmosphere, temperature of room temperature to about 400° C.-DSC, Pan Type: disposable Al pan (DSC). As a result, Tg of Compound 33 was about 151° C.

### Synthesis Example 4

Compound 42 was synthesized through Reaction Schemes 4 and 5 below:

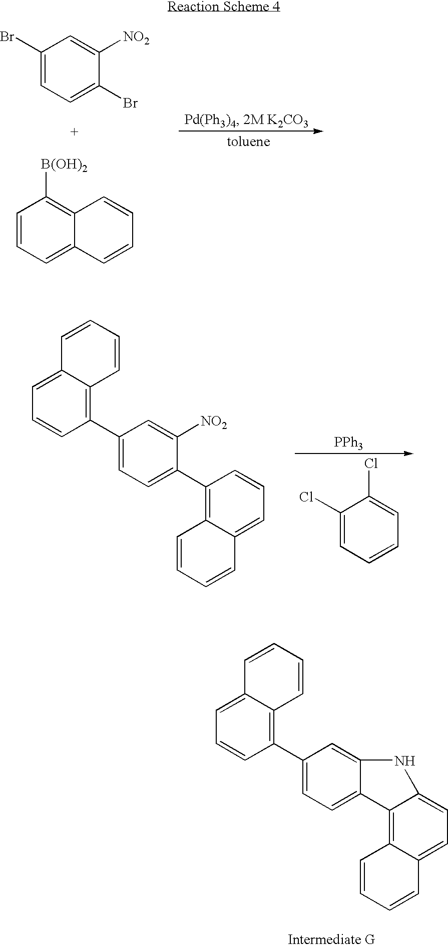

about 8.4 g (about 30 mmol) of 2,5-dibromonitrobenzene, about 10.8 g (about 62.6 mmol) of 1-naphthalene boronic acid, about 520 mg (about 0.45 mmol) of tetrakis(triphenylphosphine)palladium (Pd(PPh₃)₄ and about 63 ml (about 126 mmol) of 2M potassium carbonate (K₂CO₃) solution were respectively dissolved in about 100 ml of toluene and refluxed for about 24 hours. When the reaction was terminated, the solvent was evaporated and removed. The resultant was washed respectively with about 500 ml of ethylacetate and about 500 ml of water. Then an organic layer was collected and dried using anhydrous magnesium sulfate. Then the resultant was isolated by silica chromatography to obtain about 9.5 g of 1,1′-(2-nitro-1,4-phenylene)dinaphthalene (yield: about 84%).

About 8.0 g (21.3 mmol) of 1,1′-(2-nitro-1,4-phenylene)dinaphthalene and 14 g (about 53.3 mmol) of triphenylphosphine (PPh₃) were dissolved in about 42 ml of 1,2-dichlorobenzene and refluxed for about 24 hours. When the reaction was terminated, the resultant was isolated by silica chromatography to obtain about 4.1 g of Intermediate G (yield: about 56%).

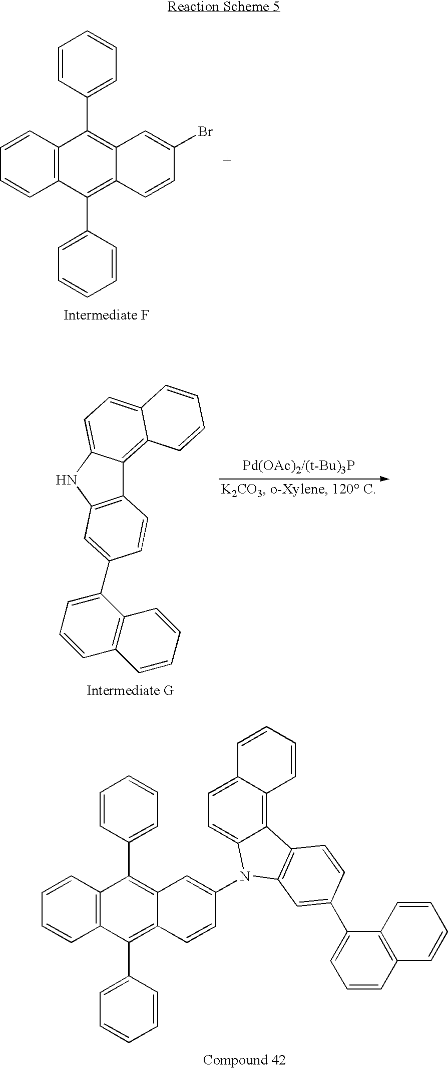

About 1.5 g (3.6 mmol) of Intermediate F, about 2.5 g (about 7.2 mmole) of Intermediate G, about 40 mg (about 0.18 mol) of Pd(OAc)₂, about 108 mg (about 0.54 mmol) of P(t-Bu)₃, and about 1.4 g (about 10 mmol) of potassium carbonate (K₂CO₃) solution were refluxed 14 ml of toluene in a nitrogen atmosphere for about 12 hours. When the reaction was terminated, the solvent was evaporated and removed. The resultant was washed respectively with about 500 ml of ethylacetate and about 500 ml of water. Then an organic layer was collected and dried using anhydrous magnesium sulfate. Then the resultant was isolated by silica chromatography to obtain about 1.3 g of Compound 42 (yield: about 60%).

¹H-NMR (CDCl₃, 300 MHz, ppm): 8.7-7.1 (m, 33H).

### Synthesis Example 5

Compound 46 was synthesized through Reaction Scheme 6 below:

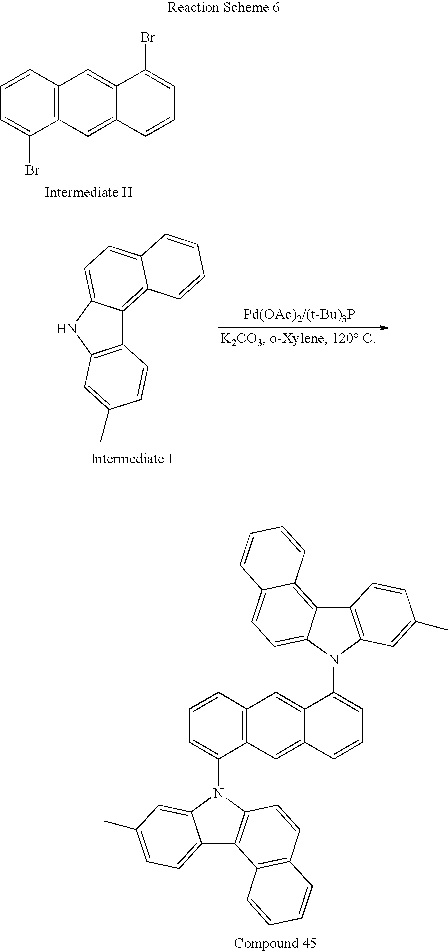

Intermediate I was synthesized using a method of synthesizing Intermediate G.

About 1.2 g (about 3.6 mmol) of Intermediate H, about 1.6 g (about 7.2 mmole) of Intermediate I, about 40 mg (about 0.18 mol) of Pd(OAc)₂, about 108 mg (about 0.54 mmol) of P(t-Bu)₃, about 1.4 g (about 10 mmol) of potassium carbonate (K₂CO₃) solution were refluxed in about 14 ml of toluene in a nitrogen atmosphere for about 12 hours. When the reaction was terminated, the solvent was evaporated and removed. The resultant was washed respectively with about 500 ml of ethylacetate and about 500 ml of water. Then an organic layer was collected and dried using anhydrous magnesium sulfate. Then the resultant was isolated by silica chromatography to obtain about 0.9 g of Compound 45 (yield: about 40%).

¹H-NMR (CDCl₃, 300 MHz, ppm):8.9-7.4 (m, 26H), 2.3 (s, 6H).

### Evaluation Example 1

### Evaluation of Emitting Properties of the Compounds (in Solution)

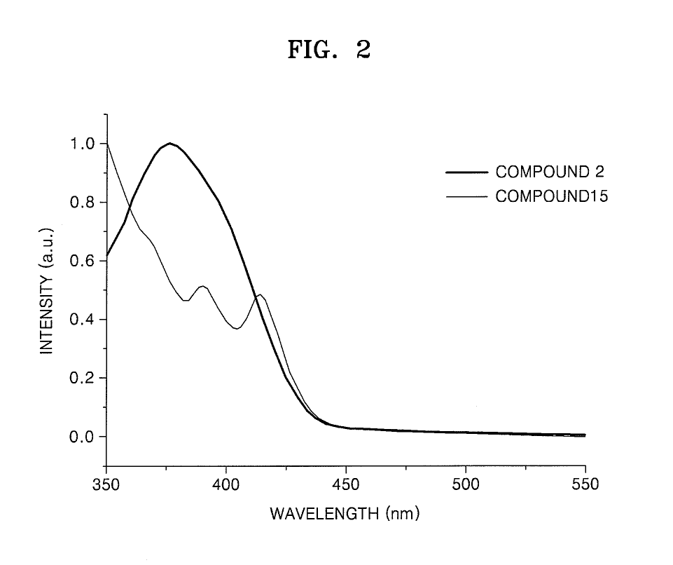

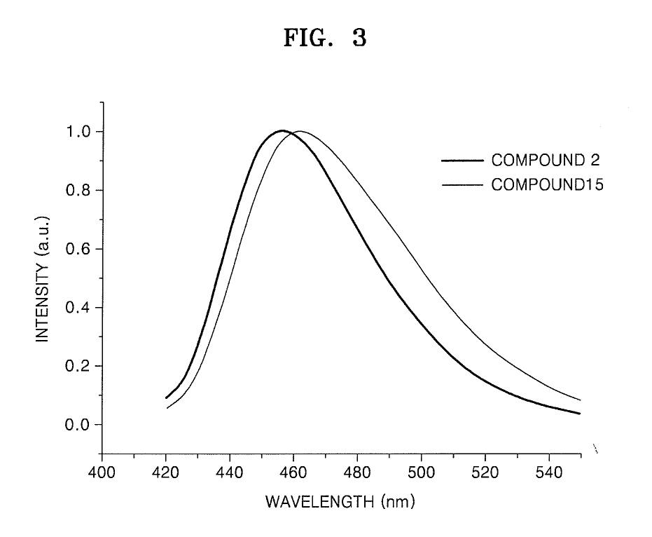

Emitting properties of the compounds described above were evaluated using UV absorption spectrum and photoluminescence (PL) spectrum. First, Compound 2 was diluted in toluene to a concentration of 0.2 mM, and a UV absorption spectrum of the solution was measured using Shimadzu UV-350 Spectrometer. The same process was repeated using Compounds 15, 33, 42, and 45. Meanwhile, Compound 2 was diluted in toluene to a concentration of about 10 mM, and Photoluminecscence (PL) of the solution was measured using an ISC PC1 Spectrofluorometer including a Xenon lamp. The results are shown in Table 1 below. The same process was repeated using Compounds 15, 33, 42 and 45. FIG. 2 is a graph illustrating UV absorption spectra of solutions of Compounds 2 and 15. FIG. 3 is a graph illustrating photoluminescence (PL) spectra of Compounds 2 and 15.

TABLE 1

| | Absorption | |
|---|---|---|
| Compound No. | wavelength (nm) | PL wavelength (nm) |
| Compound 2 | about 376 | about 456 |
| Compound 15 | about 414 | about 461 |
| Compound 33 | about 411 | about 425 |
| Compound 42 | about 330 | about 450 |
| Compound 45 | about 335 | about 438 |

Referring to Table 1, it can be seen that Compounds 2, 15, 33, 42 and 45 have emitting properties suitable for OLEDs.

### Example 1

An OLED having the following structure was manufacturing using Compound 2 as a dopant of an EML and AND as a host of the EML:

ITO(about 15 Ω/cm², about 1000 Å)/m-MTDATA(about 350 Å)/α-NPD (about 300 Å)/Compound 2: ADN (about 350 Å)/Alq₃(about 250 Å)/LiF(about 10 Å)/AI(about 2000 Å).

An ITO glass substrate was cut to a size of about 50 mm×about 50 mm×about 0.7 mm, microwave washed with acetone isopropyl alcohol for 15 minutes, microwave washed with pure water for about 15 minutes, and washed with UV ozone for about 30 minutes. Then, m-MTDATA was vacuum deposited on the substrate to form a HIL with a thickness of about 350 Å at a deposition rate of about 1 Å/sec. α-NPD was vacuum deposited on the HIL to form a HTL with a thickness of about 300 Å at a deposition rate of about 1 Å/sec. Then, Compound 2 and AND were vacuum deposited on the HTL to form an EML with a thickness of about 350 Å at deposition rates of about 5 Å/sec for Compound 2 and about 30 Å/sec for AND, wherein the doping concentration of Compound 2 was about 5 wt %. Alq₃ was vacuum deposited on the EML to form an ETL with a thickness of about 250 Å. LiF was vacuum deposited on the ETL to form an EIL with a thickness of about 10 Å and Al was vacuum deposited on the EIL to form a cathode with a thickness of about 2000 Å. An OLED manufactured as described above is referred to Sample 1.

### Example 2

An OLED (Sample 2) was manufactured in the same manner as in Example 1, except that Compound 15 was used instead of Compound 2.

### Example 3

An OLED (Sample 3) was manufactured in the same manner as in Example 1, except that Compound 33 was used instead of Compound 2.

### Example 4

An OLED (Sample 4) was manufactured in the same manner as in Example 1, except that Compound 42 was used instead of Compound 2.

### Example 5

An OLED (Sample 5) was manufactured in the same manner as in Example 1, except that Compound 45 was used instead of Compound 2.

### Comparative Example 1

An OLED (Sample 6) was manufactured in the same manner as in Example 1, except that a compound represented by Formula Z below was used instead of Compound 2.

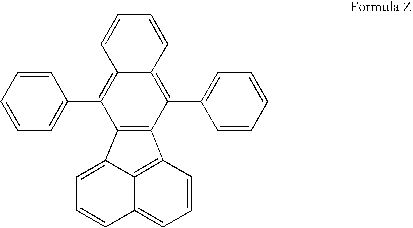

### Evaluation Example 2

### Evaluation of Properties of Samples 1 to 6

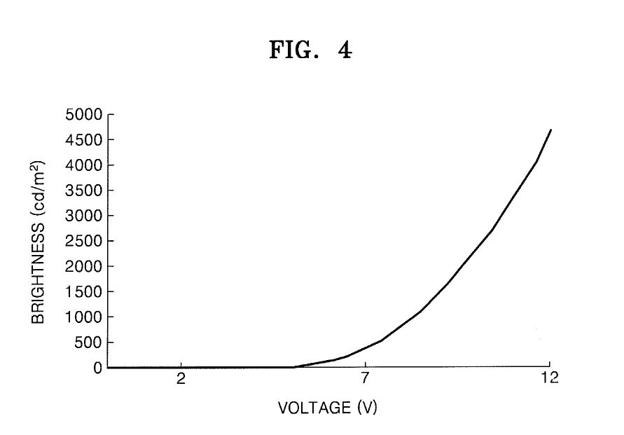

Driving voltage at about 1000 cd/m² and maximum efficiency of Samples 1 to 6 were measured using a PR650 (Spectroscan) Source Measurement Unit. The results are shown in Table 2 below. FIG. 4 is a graph illustrating voltage-brightness characteristics of Sample 1.

TABLE 2

| | Maximum | Driving voltage |
|---|---|---|
| Sample No. | efficiency (lm/W) | at 1000 cd/m² |
| Sample 1 | about 2.4 | about 8.5 |
| Sample 2 | about 2.1 | about 8.5 |
| Sample 3 | about 2.0 | about 8.5 |
| Sample 4 | about 2.3 | about 8.5 |
| Sample 5 | about 2.2 | about 8.5 |
| Sample 6 | about 1.7 | about 11 |

Referring to Table 2, Samples 1 to 5 had better maximum efficiency and driving voltage properties at about 1000 cd/m² compared to Sample 6.

Since the compounds described above are suitable for an organic layer of the OLED, an OLED having excellent performance can be manufactured using those compounds.

Having described the exemplary embodiments of the present invention, it is further noted that it is readily apparent to those of reasonable skill in the art that various modifications may be made without departing from the spirit and scope of the invention which is defined by the metes and bounds of the appended claims.

What is claimed is:

1. A fused aromatic compound represented by Formula 1 below: 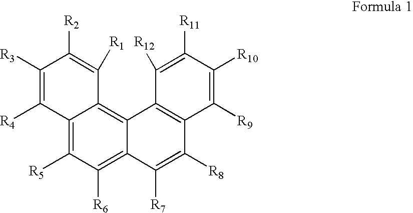
wherein R₁ to R₁₂ are each independently a hydrogen atom, a halogen atom, a cyano group, a nitro group, a hydroxyl group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₅-C₆₀ cycloalkyl group, a substituted or unsubstituted C₅-C₆₀ cycloalkenyl group, a substituted or unsubstituted C₅-C₆₀ aryl group, a substituted or unsubstituted C₁-C₆₀ heteroaryl group, or a group represented by Formula 2 below, wherein at least one of R₁ to R₁₂ is the group represented by Formula 2: 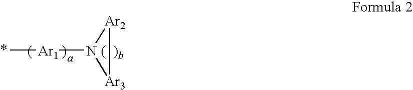
wherein Ar₁ is a substituted or unsubstituted C₅-C₆₀ arylene group;
Ar₂ and Ar₃ are each independently a substituted or unsubstituted C₅-C₆₀ aryl group;
a is an integer of 0 to 6; and
b is 0 or 1.

2. The fused aromatic compound of claim 1, represented by Formula 1a below:

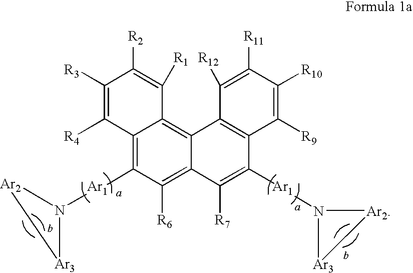

3. The fused aromatic compound of claim 1, wherein R₁ to R₁₂ are each independently a hydrogen atom, a C₁-C₁₀ alkyl group, a C₂-C₁₀ alkenyl group, a C₅-C₁₄ aryl group, a C₅-C₁₄ aryl group substituted with at least one C₁-C₁₀ alkyl group, a C₅-C₁₄ aryl group substituted with at least one —Si(R₂₁)(R₂₂)(R₂₃), or the group represented by Formula 2;
Ar₁ is a C₅-C₁₄ arylene group, a C₅-C₁₄ arylene group substituted with at least one C₁-C₁₀ alkyl group, a C₅-C₁₄ arylene group substituted with at least one C₅-C₁₄ aryl group, or a C₅-C₁₄ arylene group substituted with at least one —Si(R₂₁)(R₂₂)(R₂₃); and
Ar₂ and Ar₃ are each independently a C₅-C₁₄ aryl group, a C₅-C₁₄ aryl group substituted with at least one C₁-C₁₀ alkyl group, a C₅-C₁₄ aryl group substituted with at least one C₅-C₁₄ aryl group, or a C₅-C₁₄ aryl group substituted with at least one —Si(R₂₁)(R₂₂)(R₂₃),
wherein R₂₁ to R₂₃ are each independently a hydrogen atom, a C₁-C₁₀ alkyl group, or a substituted or unsubstituted C₅-C₁₄ aryl group.

4. The fused aromatic compound of claim 1,
wherein the group represented by 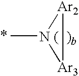
in Formula 2 is represented by one of Formulae 2a to 2k below: 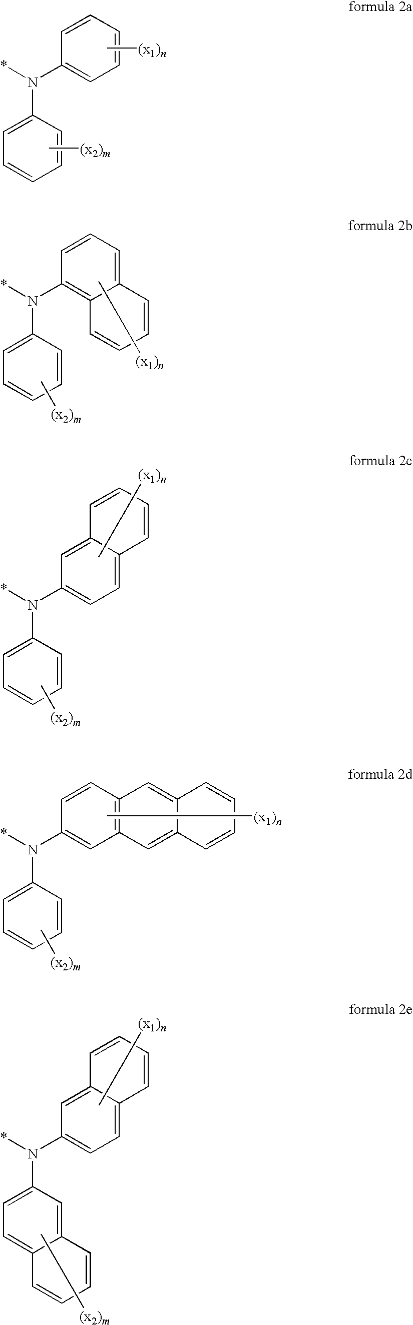 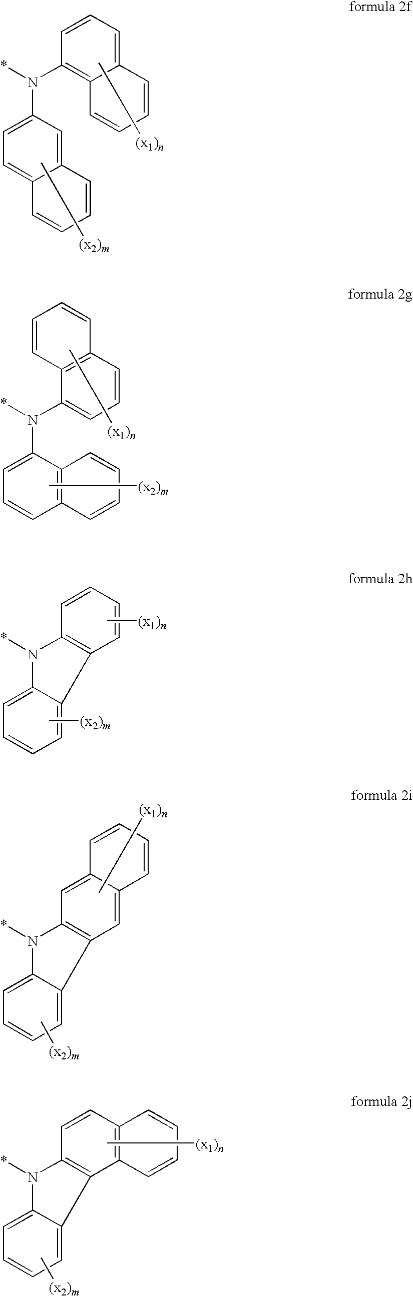 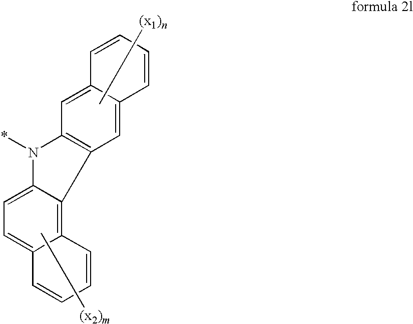
wherein X₁ and X₂ are each independently a C₁-C₁₀ alkyl group, a C₅-C₁₄ aryl group, or —Si(R₂₁)(R₂₂)(R₂₃);
n and m are each independently an integer of 0-6; and
* is a binding site for Ar₁ or a benzophenanthrene ring.

5. A fused aromatic compound represented by Formula 3 below: 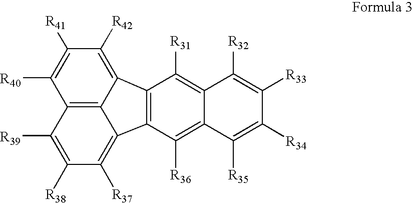
wherein R₃₁ to R₃₆ are each independently a hydrogen atom, a halogen atom, a cyano group, a nitro group, a hydroxyl group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₅-C₆₀ cycloalkyl group, a substituted or unsubstituted C₅-C₆₀ cycloalkenyl group, a substituted or unsubstituted C₅-C₆₀ aryl group, a substituted or unsubstituted C₁-C₆₀ heteroaryl group, a group represented by Formula 4 below, or a group represented by Formula 5 below; and
R₃₇ to R₄₂ are each independently a hydrogen atom or the group represented by Formula 5; 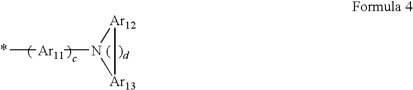
*-(Ar₁₄)_{X}-Q₁ Formula 5
wherein Ar₁₁ and Ar₁₄ are each independently a C₅-C₆₀ arylene group, or a C₅-C₆₀ arylene group substituted with at least one selected from the group consisting of a halogen atom, a cyano group, a nitro group, a hydroxyl group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₅-C₆₀ cycloalkyl group, a substituted or unsubstituted C₅-C₆₀ cycloalkenyl group, a substituted or unsubstituted C₅-C₆₀ aryl group, a substituted or unsubstituted C₁-C₆₀ heteroaryl group, and —Si(R₄₄)(R₄₅)(R₄₆);
Ar₁₂ and Ar₁₃ are each independently a C₅-C₁₄ aryl group, a C₅-C₁₄ aryl group substituted with at least one C₁-C₁₀ alkyl group, a C₅-C₁₄ aryl group substituted with at least one C₅-C₁₄ aryl group, or a C₅-C₁₄ aryl group substituted with at least one —Si(R₄₄)(R₄₅)(R₄₆); and
Q₁ is a hydrogen atom or —Si(R₄₇)(R₄₈)(R₄₉),
wherein R₄₄ to R₄₉ are each independently a hydrogen atom, a C₁-C₁₀ alkyl group, or a C₅-C₁₄ aryl group;
c is an integer of 1 to 6;
d is 0 or 1; and
x is an integer of 0 to 6,
1) except for when all of R₃₁ to R₄₂ are hydrogen atoms,
2) when all of R₃₇ to R₄₂ are hydrogen atoms, R₃₁ to R₃₆ are each independently a hydrogen atom, a group represented by Formula 4, or a group represented by Formula 5, provided that at least one of R₃₁ to R₃₆ is the group represented by Formula 4, or the group represented by Formula 5 wherein Q₁ is —Si(R₄₇)(R₄₈)(R₄₉), and
3) when all of R₃₁ to R₃₆ are hydrogen atoms, at least one of R₃₇ to R₄₂ is the group represented by Formula 5 wherein Q₁ is —Si(R₄₇)(R₄₈)(R₄₉).

6. The fused aromatic compound of claim 6, represented by Formula 3a below: 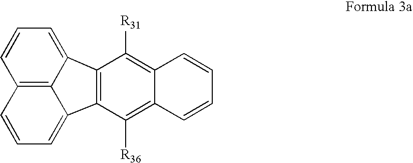
wherein R₃₁ and R₃₆ are each independently a hydrogen atom, the group represented by Formula 4, or the group represented by Formula 5, provided that at least one of R₃₁ and R₃₆ is the group represented by Formula 4 or the group represented by Formula 5 wherein Q₁ is —Si(R₄₇)(R₄8)(R₄₉).

7. The fused aromatic compound of claim 6, represented by Formula 3b below:

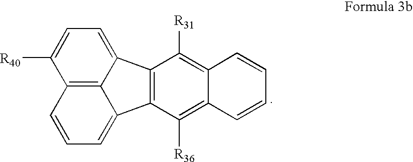

8. The fused aromatic compound of claim 6, wherein R₃₁ to R₃₆ are each independently a hydrogen atom, a halogen atom, a cyano group, a nitro group, a hydroxyl group, a C₁-C₁₀ alkyl group, a C₂-C₁₀ alkenyl group, a C₂-C₁₀ alkynyl group, a C₁-C₁₀ alkoxy group, a C₅-C₁₄ aryl group, the group represented by Formula 4, or the group represented by Formula 5;
Ar₁₁ and Ar₁₄ are each independently a C₅-C₁₄ arylene group, a C₅-C₁₄ arylene group substituted with at least one C₁-C₁₀ alkyl group, a C₅-C₁₄ arylene group substituted with at least one C₅-C₁₄ aryl group, or a C₅-C₁₄ arylene group substituted with at least one —Si(R₄₄)(R₄₅)(R₄₆);
Ar₁₂ and Ar₁₃ are each independently a C₅-C₁₄ aryl group, a C₅-C₁₄ aryl group substituted with at least one C₁-C₁₀ alkyl group, a C₅-C₁₄ aryl group substituted with at least one C₅-C₁₄ aryl group, or a C₅-C₁₄ aryl group substituted with at least one —Si(R₄₄)(R₄₅)(R₄₆); and
Q₁ is a hydrogen atom or —Si(R₄₇)(R₄₈)(R₄₉),
wherein R₄₄ to R₄₉ are each independently a hydrogen atom, a C₁-C₁₀ alkyl group, a C₂-C₁₀ alkenyl group, or a C₅-C₁₄ aryl group.

9. The fused aromatic compound of claim 6,
wherein the group represented by 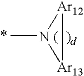
in Formula 4 is represented by one of Formulae 4a to 4k below: 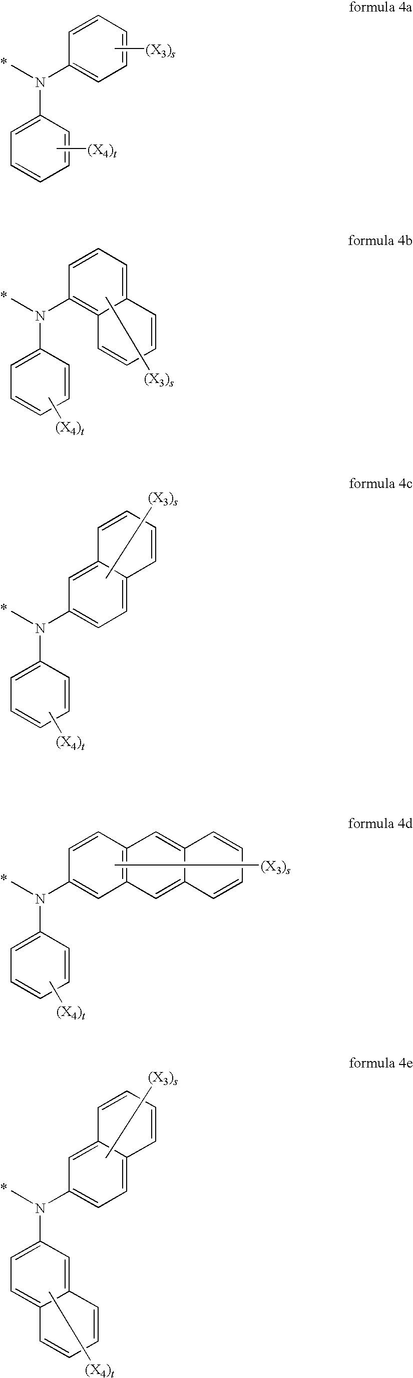 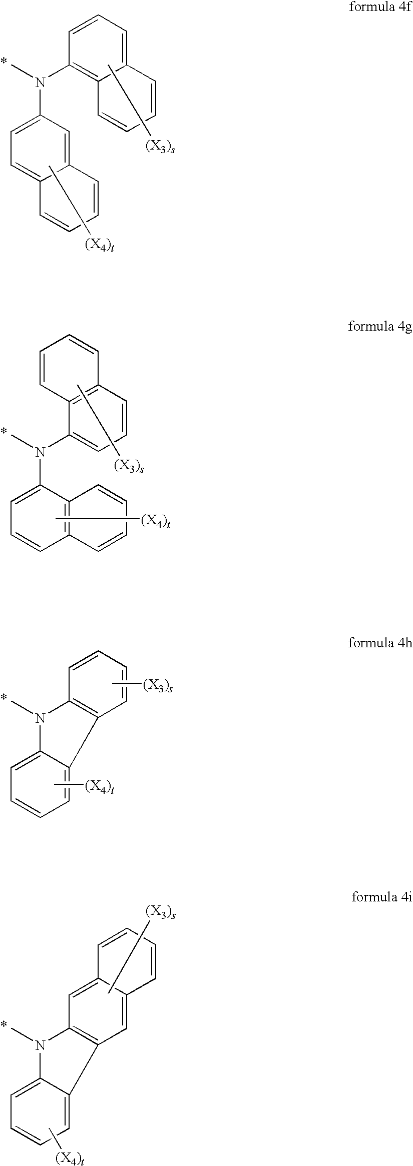 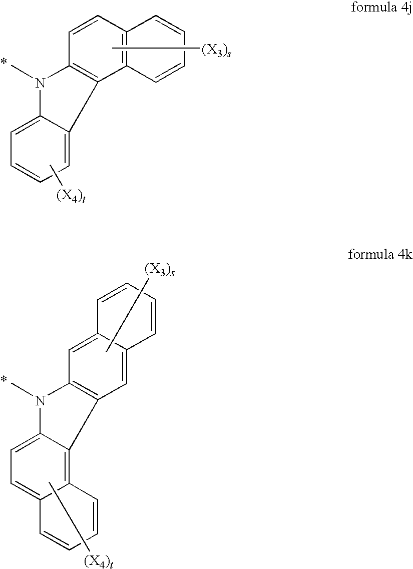
wherein X₃ and X₄ are each independently a C₁-C₁₀ alkyl group, a C₅-C₁₄ aryl group, or —Si(R₄₄)(R₄₅)(R₄₆);
s and t are each independently an integer of 0 to 6; and
* is a binding site for a benzofluoranthene ring.

10. The fused aromatic compound of claim 6, wherein the group represented by Formula 5 is represented by one of Formulae 5a to 5f below: 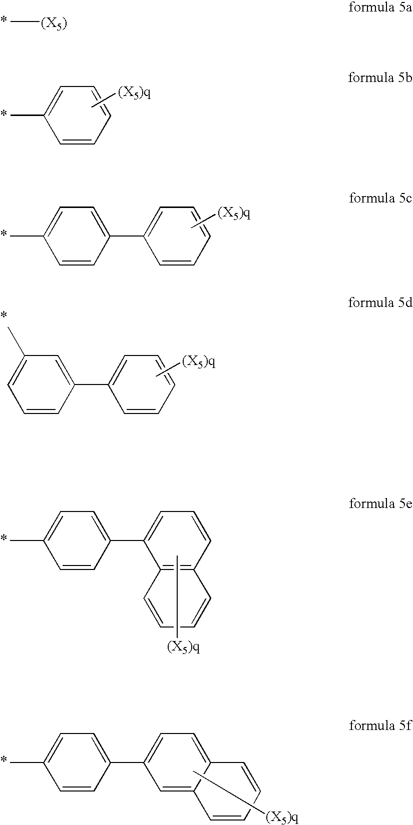
wherein X₅ is a C₁-C₁₀ alkyl group, a C₅-C₁₄ aryl group, or —Si(R₄₇)(R₄₈)(R₄₉); and
Q is an integer of 0 to 6.

11. A fused aromatic compound represented by Formula 6 below: 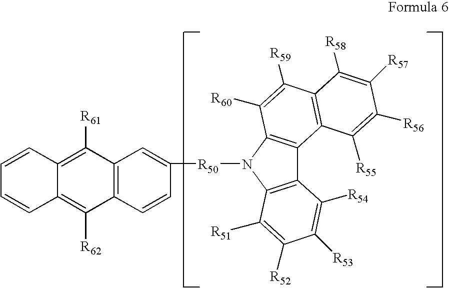
wherein one of R₅₀ to R₆₀ is bonded to an anthracene ring of Formula 6;
R₅₀ to R₆₀ are each independently a hydrogen atom, a halogen atom, a cyano group, a nitro group, a hydroxyl group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₅-C₆₀ cycloalkyl group, a substituted or unsubstituted C₅-C₆₀ cycloalkenyl group, a substituted or unsubstituted C₅-C₆₀ aryl group, a substituted or unsubstituted C₁-C₆₀ heteroaryl group, or —Si(R₆₅)(R₆₆)(R₆₇), wherein at least two adjacent groups among R₅₀ to R₆₀ are bonded to each other to form a ring;
R₆₁ and R₆₂ are each independently a substituted or unsubstituted C₁-C₆₀ alkyl group, or a phenyl group; and
R₆₅ to R₆₇ are each independently a hydrogen atom, a C₁-C₁₀ alkyl group, a C₂-C₁₀ alkenyl group, or a C₅-C₁₄ aryl group.

12. The fused aromatic compound of claim 11, represented by Formula 6a or 6b below:

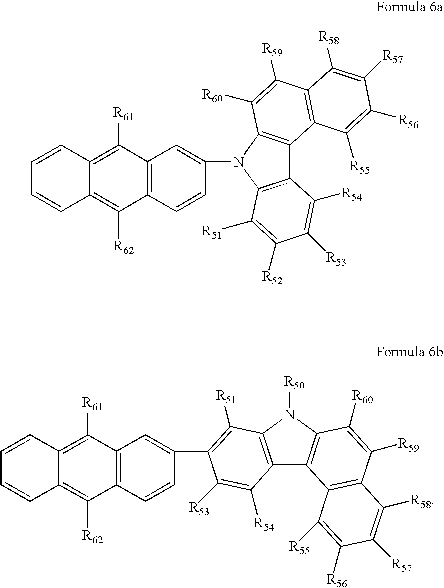

13. The fused aromatic compound of claim 11, wherein R₅₀ to R₆₀ are each independently a hydrogen atom, a C₁-C₁₀ alkyl group, or a C₅-C₁₄ aryl group.

14. A fused aromatic compound represented by Formula 7 below: 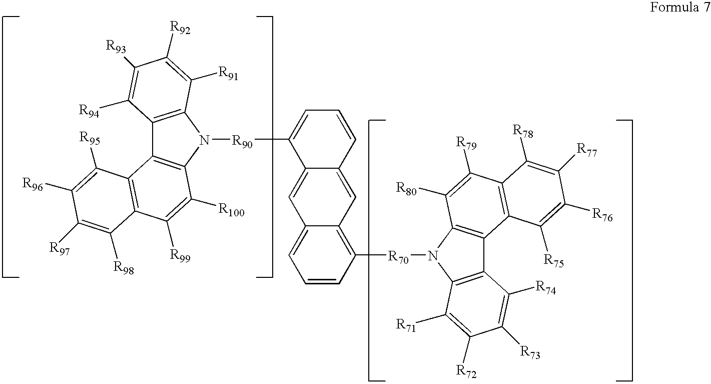
wherein one of R₇₀ to R₈₀ and one of R₉₀ to R₁₀₀ are bonded to an anthracene ring of Formula 7;
R₇₀ to R₈₀ and R₉₀ to R₁₀₀ are each independently a hydrogen atom, a halogen atom, a cyano group, a nitro group, a hydroxyl group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₅-C₆₀ cycloalkyl group, a substituted or unsubstituted C₅-C₆₀ cycloalkenyl group, a substituted or unsubstituted C₅-C₆₀ aryl group, a substituted or unsubstituted C₁-C₆₀ heteroaryl group, or —Si(R₁₀₁)(R₁₀₂)(R₁₀₃), wherein at least two adjacent groups among R₇₀ to R₈₀ and R₉₀ to R₁₀₀ are bonded to each other to form a ring; and
R₁₀₁ to R₁₀₃ are each independently a hydrogen atom, a C₁-C₁₀ alkyl group, a C₂-C₁₀ alkenyl group, or a C₅-C₁₄ aryl group.

15. The fused aromatic compound of claim 14, represented by Formula 7a or 7b below:

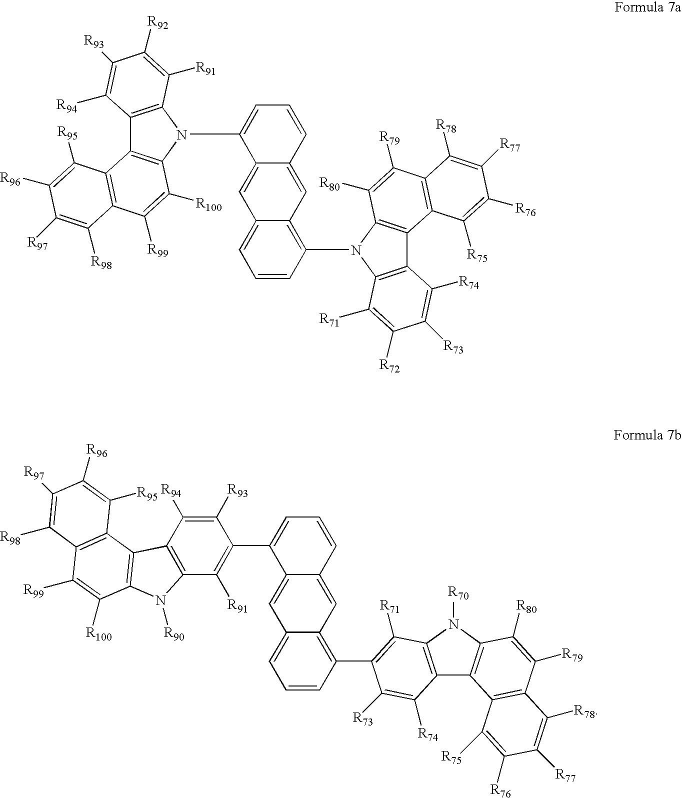

16. The fused aromatic compound of claim 14, wherein R₇₀ to R₈₀ and R₉₀ to R₁₀₀ are each independently a hydrogen atom, a C₁-C₁₀ alkyl group, or a C₅-C₁₄ aryl group.

17. An organic light emitting diode (OLED) comprising a substrate, a first electrode, a second electrode, and an organic layer interposed between the first electrode and the second electrode, wherein the organic layer comprises the compound of claim 1.

18. An organic light emitting diode (OLED) comprising a substrate, a first electrode, a second electrode, and an organic layer interposed between the first electrode and the second electrode, wherein the organic layer comprises the compound of claim 5.

19. An organic light emitting diode (OLED) comprising a substrate, a first electrode, a second electrode, and an organic layer interposed between the first electrode and the second electrode, wherein the organic layer comprises the compound of claim 11.

20. An organic light emitting diode (OLED) comprising a substrate, a first electrode, a second electrode, and an organic layer interposed between the first electrode and the second electrode, wherein the organic layer comprises the compound of claim 14.
